(19) United States
(12) Patent Application Publication
(10) Pub. No.: US 2020/0216867 A1
(43) Pub. Date: Jul. 9, 2020

(54) METHOD FOR PRODUCING L-TRYPTOPHAN USING IMPROVED STRAINS OF THE ENTEROBACTERIACEAE FAMILY

(71) Applicant: Evonik Operations GmbH, Essen (DE)

(72) Inventors: Mechthild Rieping, Bielefeld (DE); Silke Jerrentrup, Bielefeld (DE); Nicole Dusch, Werther (DE)

(21) Appl. No.: 16/734,362

(22) Filed: Jan. 5, 2020

(30) Foreign Application Priority Data
Jan. 7, 2019 (EP) 19150500.7
Jan. 30, 2019 (CN) 201910090877.7

(51) Int. Cl. C12P 13/22; C12N 15/71

(52) U.S. Cl. CPC C12P 13/227; C12R 1/19; C12N 15/71

(57) ABSTRACT

The invention provides a method of producing L-tryptophan, the method comprising culturing a L-tryptophan producing microorganism belonging to the Enterobacteriaceae family in a fermentation medium; wherein the L-tryptophan producing microorganism has been modified by enhancing the expression level of the mdfA gene or by enhancing the expression level of an mdfA allele.

### CROSS REFERENCE TO RELATED APPLICATIONS

The present application claims priority under 35 USC § 119 to European application EP19150500.7, filed on Jan. 7, 2019 (the contents of which is incorporated herein by reference in its entirety) and to Chinese application, CN 201910090877.7, filed Jan. 30, 2019 (the contents of which is also incorporated herein by reference in its entirety).

### FIELD OF THE INVENTION

The present invention pertains to a new method for the fermentative production of L-tryptophan using a modified microorganism of the Enterobacteriaceae family, in which the expression level of the mdfA gene is enhanced.

### BACKGROUND OF THE INVENTION

L-tryptophan is used in human medicine and in the pharmaceutical industry, in food industry and in animal nutrition.

L-tryptophan can be produced by fermentation of Enterobacteriaceae strains, especially Escherichia coli (E. coli) and Serratia marcescens. Because of the great significance, work is constantly being done on improving the methods of production.

Methodological improvements may concern measures relating to fermentation technology, such as, for example, stirring and oxygen supply, or the composition of the nutrient media, such as, for example, selection of the sugar used or the sugar concentration during the fermentation, or the working-up to the product form, for example by means of ion exchange chromatography, or the intrinsic performance properties of the microorganism itself.

In wild-type strains, strict regulatory mechanisms prevent metabolic products such as amino acids from being produced in excess of what is needed by said strains and from being released into the medium. The construction of amino acid-overproducing strains therefore requires, from a manufacturer's point of view, these metabolic regulations to be overcome.

Methods of mutagenesis, selection and mutant choice are used for removing said control mechanisms and improving the performance properties of these microorganisms.

Owing to the complexity of the biosynthesis pathway of aromatic L-amino acids like L-tryptophan and owing to the interconnection thereof with many other metabolic pathways in the cell, it is not always possible to predict which genetic variations or modifications of a strain can achieve an improved production of L-tryptophan.

The multidrug transporter MdfA is known as an efflux pump driven by the proton motive force (Lewinson et al., Proc. Natl. Acad. Sci. U.S.A. 100 (4) 1667-1672). Its additional function as a Na⁺/H⁺ antiporter and its involvement in the production and accumulation of amino acids, such as L-lysine, L-arginine, L-threonine or L-histidine, has been reported in WO 2007/069782 A1. Therein, a bacterium of the Enterobacteriaceae family with increased expression level of a gene selected from the group consisting of nhaA, nhaB, nhaR, chaA, mdfA and combinations thereof is disclosed.

However, WO 2007/069782 A1 is silent on the influence of an enhanced mdfA expression level on L-tryptophan productivity.

In view of the above, it was a remaining need to further optimize especially those strains of the Enterobacteriaceae family that are suitable for producing L-tryptophan, and to provide improved methods for the fermentative production of L-tryptophan, using these microorganisms of the Enterobacteriaceae family. Further, it was particularly desirable to establish new techniques enabling the modulation of the expression level of genes involved in transport processes of aromatic compounds as toxicity caused by (even low amounts of) aromatic amino acid derivates and intermediates requires effective transport systems. However, the gene expression levels should be adapted to the specific needs of the abundance of transporter proteins in the cell membrane without affecting cellular fitness and general production capacities.

### SUMMARY OF THE INVENTION

The present invention provides a method of producing L-tryptophan, the method comprising culturing a L-tryptophan producing microorganism belonging to the Enterobacteriaceae family in a fermentation medium; wherein the L-tryptophan producing microorganism has been modified by enhancing the expression level of the mdfA gene or by enhancing the expression level of an mdfA allele.

Further, the present invention pertains to a method for producing an L-tryptophan producing microorganism by transformation, transduction or conjugation, wherein the transformation, transduction or conjugation is done using a vector comprising:
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2;
g) a polynucleotide encoding a protein comprising an amino acid sequence which includes substitution, deletion, insertion or addition of 1 to 20 amino acids in SEQ ID NO.:2;

and comprising a promoter regulating the expression of polynucleotides a) to g).

In addition, the invention provides a L-tryptophan producing microorganism of the Enterobacteriaceae family, wherein the microorganism has been modified by enhancing the expression level of the mdfA gene or by enhancing the expression level of an mdfA allele.

Finally, the invention is directed to the use of the aforementioned microorganism for producing L-tryptophan or, alternatively, for producing a feedstuff additive containing L-tryptophan.

### DETAILED DESCRIPTION OF THE INVENTION

In the work forming the basis of the present invention, it was surprisingly found that the L-tryptophan productivity in a L-tryptophan producing strain of the Enterobacteriaceae family may be significantly increased by modulating the expression level of the mdfA gene or by modulating the expression level of a mdfA allele, respectively in an appropriate way.

More specifically, the present invention provides a method of producing L-tryptophan, the method comprising culturing a L-tryptophan producing microorganism belonging to the Enterobacteriaceae family in a fermentation medium; wherein the L-tryptophan producing microorganism has been modified by enhancing the expression level of the mdfA gene or by enhancing the expression level of an mdfA allele.

For the sake of clarity and completeness, the coding region of the mdfA gene is depicted in SEQ ID NO.:1. The amino acid sequence of MdfA is depicted in SEQ ID NO.:2.

An allele is a variant of a given gene.

The mdfA allele according to the present invention may be a polynucleotide selected from the group consisting of:
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80%, at least 85%, or at least 90%, preferably at least 95% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2;
g) a polynucleotide encoding a protein comprising an amino acid sequence which includes substitution, deletion, insertion or addition of 1 to 20 amino acids in SEQ ID NO.:2.

The terms “enhancing’ or ‘enhanced’ or ‘overexpressed’ or ‘increased expression level’ ‘enhanced expression level’ or ‘overexpression” are used interchangeably in this text and have similar meaning. These terms, in this context, describe the increase in the intracellular activity of an enzymatic activity which is encoded by the corresponding DNA, for example by increasing the number of copies of the gene, using a stronger promoter or using an allele with increased activity and possibly combining these measures.

The expression level of the mdfA gene or of the mdfA allele may be enhanced by increasing the copy number of the gene or allele by at least 1.

To increase the expression level of a gene/allele, copies of genes/alleles are preferably added chromosomally. Chromosomally there may be one or several extra copies on the genome that may be introduced by methods of recombination known to the man skilled in the art. Extra-chromosomal genes may be carried by different types of plasmids or bacterial artificial chromosomes that differ with respect to their origin of replication and thus their copy number in the cell. They may be present as 1-5 copies, about 20 or up to 500 copies, corresponding to low copy number plasmids with tight replication, medium copy number plasmids, or high copy number plasmids.

The copy number of the mdfA gene or of the mdfA allele may be increased by at least 1 by integrating the gene into the chromosome of the microorganism.

The expression level of the mdfA gene or of the mdfA allele may also be enhanced by modifying the expression regulatory sequence of the gene. As indicated before, the mdfA gene or mdfA allele may be combined with a stronger promoter. These promoters may be inducible; they may be homologous or heterologous. The man skilled in the art knows which promoters are the most convenient.

When genes are organized in an operon, it is possible to enhance their expression level by adding one supplementary copy of these genes under control of a single promoter. Expression may also be enhanced by replacing the chromosomal wild-type promoter with an artificial promoter stronger than the wild-type promoter. The expert in the field knows how to determine promoter strength.

The microorganisms according to the present invention and used in the method according to the present invention, respectively, are representatives of the Enterobacteriaceae family, selected from the genera Escherichia, Erwinia and Providencia. The genus Escherichia is preferred. The species Escherichia coli must be mentioned in particular. Preferably, a microorganism having an increased expression of the Trp operon is used.

The inventors have surprisingly found that a particularly strong L-tryptophan productivity may be achieved in a L-tryptophan producing strain of the Enterobacteriaceae, in particular of Escherichia coli, in case the mdfA gene or the mdfA allele is integrated into specific chromosomal or extra-chromosomal loci. More specifically, the inventors have found that using the specific chromosomal or extra-chromosomal loci for mdfA integration enables the modulation of the expression level of mdfA in a way that improves the capacity of fermentative production of L-tryptophan when commercially relevant concentrations accumulate in the fermentation broth.

As used in the context of the present invention, the term “modulation of expression level” refers to setting a balanced expression level leading to an optimized yield of the amino acid (L-tryptophan) product.

The expression of a stable integrated gene is strongly influenced by regulatory sequences in the chromosomal environment of the integration site, e.g. combined effects of the start codon and the flanking regions have been described (Stenstrom et al., Gene 273(2):259-65 (2001); Hui et al., EMBO Journal 3(3):623-9 (1984)).

In one embodiment, a copy of the mdfA gene or the mdfA allele with native expression signals is integrated in the chromosomal mtr locus, whereby the mtr gene is deleted simultaneously. Preferably, the mtr promoter remains intact, as indicated in SEQ ID NO.:25, i.e. the flanking regions probably regulates the expression of the mdfA gene or the mdfA allele.

In addition or as an alternative to the above, a copy of the mdfA gene or of the mdfA allele may be integrated in the chromosomal aroP locus, whereby the aroP gene is deleted simultaneously. Preferably, the aroP promoter remains intact, i.e. the flanking regions probably regulates the expression of the mdfA gene or the mdfA allele.

Accordingly, the mdfA expression level might be adapted to the specific needs of the abundance of MdfA proteins in the cell membrane by using different integration loci, because regulatory sequences of the flanking regions might have impact.

The microorganisms according to the invention and used in the method of the present invention, respectively, can be produced using the above-described methods for transformation, transduction or conjugation. Preferably, the transformation, transduction or conjugation is done using a vector comprising
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80%, at least 85%, or at least 90%, preferably at least 95% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2;
g) a polynucleotide encoding a protein comprising an amino acid sequence which includes substitution, deletion, insertion or addition of 1 to 20 amino acids in SEQ ID NO.:2;

and comprising a promoter regulating the expression of polynucleotides a) to g).

To produce the inventive strains of the Enterobacteriaceae family, preference is given to using strains (starting strains or parent strains) already having the capability of enriching L-tryptophan in the cell and/or of secreting it into the nutrient medium surrounding the cell or accumulating it in the fermentation broth. The expression “produce” can also be used for this.

More particularly, the strains used for the measures of the invention have the capability of enriching or of accumulating in the cell and/or in the nutrient medium or the fermentation broth≥(at least) 0.25 g/l, ≥0.5 g/l, ≥1.0 g/l, ≥1.5 g/l, ≥2.0 g/l, ≥4 g/l, ≥10 g/l, ≥20 g/l, ≥30 g/l or ≥50 g/l of L-tryptophan, in ≤(at most) 120 hours, ≤96 hours, ≤48 hours, ≤36 hours, ≤24 hours or ≤12 hours. Here, the strains can be strains which have been produced by mutagenesis and selection, by recombinant DNA techniques or by a combination of both methods.

The preferably recombinant microorganisms containing the nucleotide sequence according to the present invention can produce L-tryptophan, from glucose, sucrose, lactose, fructose, maltose, molasses, possibly starch, possibly cellulose or from glycerol and ethanol, possibly also from mixtures.

Examples of strains of the genus Escherichia, in particular of the species Escherichia coli, that are suitable for the parent strain and produce or secrete L-tryptophan are:

| Escherichia coli SV164(pGH5) | (WO 94/08031) |
|---|---|
| E. coli AGX17(pGX44) (NRRL B-12263) | (U.S. Pat. No. |
| | 4,371,614) |
| E. coli AGX6(pGX50)aroP (NRRL B-12264) | (U.S. Pat. No. |
| | 4,371,614) |
| Escherichia coli AGX17/pGX50, pACKG4-pps | (WO 97/08333) |
| Escherichia coli ATCC 31743 | (CA 1182409) |
| E. coli C534/pD2310, pDM136 (ATCC 39795) | (WO8 7/01130) |
| Escherichia coli JB102/p5LRPS2 | (U.S. Pat. No. |
| | 5,939,295) |

L-Tryptophan-producing or L-tryptophan-secreting strains from the Enterobacteriaceae family preferably have, inter alia, one or more of the genetic or phenotypic features selected from the group consisting of: resistance to 5-methyl-DL-tryptophan, resistance to 5-fluoro-tryptophan, resistance to 4-methyl-DL-tryptophan, resistance to 6-methyl-DL-tryptophan, resistance to 4-fluoro-tryptophan, resistance to 6-fluoro-tryptophan, resistance to anthranilate, resistance to tryptazan, resistance to indole, resistance to indoleacrylic acid, need for phenylalanine, need for tyrosine, possibly capacity for sucrose utilization, enhancement of the tryptophan operon, preferably anthranilate synthase, preferably the feedback-resistant form, a partially defective tryptophanyl-tRNA synthase, an attenuated tryptophan uptake, a defective tryptophanase, inactivated repressor proteins, enhancement of serine biosynthesis, enhancement of phophoenolpyruvate synthesis, enhancement of D-erythrose 4-phosphate synthesis, enhancement of 3-deoxy-D-arabino-heptulosonate 7-phosphate (DHAP) synthesis, enhancement of chorismate biosynthesis.

Furthermore, for the production of L-tryptophan with strains of the Enterobacteriaceae family it may be advantageous to additionally enhance one or more enzymes of the known biosynthesis pathways or enzymes of anaplerotic metabolism or enzymes for the production of reduced nicotinamide adenine dinucleotide phosphate or enzymes of glycolysis or PTS enzymes or enzymes of sulphur metabolism. The use of endogenous genes is generally preferred.

Furthermore, for the production of L-tryptophan, it may be advantageous to additionally switch off undesired secondary reactions (Nakayama: “Breeding of Amino Acid Producing Microorganisms”, in: Overproduction of Microbial Products, Krumphanzl, Sikyta, Vanek (eds.), Academic Press, London, U K, 1982).

In accordance with the above, the present invention further provides an L-tryptophan producing microorganism of the Enterobacteriaceae family, whereby the microorganism has been modified by enhancing the expression level of the mdfA gene or by enhancing the expression level of an mdfA allele.

In one embodiment, the mdfA allele is a polynucleotide selected from the group consisting of:
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80%, at least 85%, or at least 90%, preferably at least 95% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2;
g) a polynucleotide encoding a protein comprising an amino acid sequence which includes substitution, deletion, insertion or addition of 1 to 20 amino acids in SEQ ID NO.:2.

For enhancing the mdfA expression, the mdfA gene/allele is preferably integrated in the chromosomal mtr locus. In one specific embodiment, a copy of the mdfA gene or of the mdfA allele is integrated in the mtr locus with simultaneous deletion of the mtr gene. Optionally, the chromosomal environment of the mtr locus remains intact and regulates the expression of the mdfA gene or the mdfA allele integrated in the mtr locus.

The aforementioned L-tryptophan producing microorganism may be used for producing L-tryptophan or, alternatively, for producing a feedstuff additive containing L-tryptophan.

The microorganisms according to the invention and used in the method according to the present invention, respectively are cultivated in the batch process, in the fed-batch process, in the repeated fed-batch process or in a continuous process (DE102004028859.3 or U.S. Pat. No. 5,763,230). Overviews of such processes are available in the textbook by Chmiel (Bioprozesstechnik 1. Einführung in die Bioverfahrenstechnik [Bioprocess technology 1. Introduction to bioprocess technology] (Gustav Fischer Verlag, Stuttgart, 1991)) or in the textbook by Storhas (Bioreaktoren und periphere Einrichtungen [Bioreactors and peripheral devices] (Vieweg Verlag, Braunschweig/Wiesbaden, 1994)).

In the case of a batch process, all starting materials, with some exceptions, such as oxygen and pH correcting agent for example, are initially charged in the form of a batch and the microorganism is cultured in the medium obtained.

In the case of a fed-batch process, the microorganism is initially cultured by means of a batch process (batch phase). This is followed by adding to the culture in a continuous or discontinuous manner a starting material essential for the production of the product, also multiple starting materials if necessary (feed phase).

In the case of a repeated fed-batch process, what is involved is a fed-batch process in which, after completion of fermentation, a portion of the fermentation broth obtained serves as inoculum for starting a fresh repeated fed-batch process. This cycle can be repeated multiple times if necessary. Repeated fed-batch processes are, for example, described in WO 02/18543 and WO 05/014843.

In the case of a continuous process, a batch or fed-batch process is followed by continuously adding one or more, possibly all, starting materials to the culture and simultaneously removing fermentation broth. Continuous processes are, for example, described in the patent specifications U.S. Pat. No. 5,763,230, WO 05/014840, WO 05/014841 and WO 05/014842. The culture medium has to satisfy the demands of the particular strains in a suitable manner.

Culture media of different microorganisms are described in the handbook “Manual of Methods for General Bacteriology” of the American Society for Bacteriology (Washington D.C., USA, 1981). The terms culture medium, fermentation medium and nutrient medium or medium are interchangeable.

In general, a culture medium contains, inter alia, one or more carbon source(s), nitrogen source(s) and phosphorus source(s).

It is possible to use, as carbon source, sugars and carbohydrates such as, for example, glucose, sucrose, lactose, fructose, maltose, molasses, starch and possibly cellulose, oils and fats such as, for example, soybean oil, sunflower oil, arachis oil and coconut fat, fatty acids such as, for example, palmitic acid, stearic acid and linoleic acid, alcohols such as, for example, glycerol and ethanol and organic acids such as, for example, acetic acid. These substances may be used individually or as a mixture.

It is possible to use, as nitrogen source, organic nitrogen-containing compounds such as peptones, yeast extract, meat extract, malt extract, corn steep liquor, soybean flour and urea, or inorganic compounds such as ammonium sulphate, ammonium chloride, ammonium phosphate, ammonium carbonate and ammonium nitrate. The nitrogen sources may be used individually or as a mixture.

It is possible to use, as phosphorus source, phosphoric acid, potassium dihydrogen phosphate or dipotassium hydrogen phosphate or the corresponding sodium-containing salts.

Furthermore, the culture medium must contain salts of metals, such as magnesium sulphate or iron sulphate for example, that are necessary for growth. Finally, essential growth substances such as amino acids and vitamins may be used in addition to the substances mentioned above. Moreover, suitable precursors may be added to the culture medium. The aforementioned starting materials may be added to the culture in the form of a single batch or be appropriately fed in during cultivation.

The fermentation is generally carried out at a pH of from 5.5 to 9.0, more particularly from 6.0 to 8.0. To control the pH of the culture, appropriate use is made of basic compounds such as sodium hydroxide, potassium hydroxide, ammonia or aqueous ammonia or acidic compounds such as phosphoric acid or sulphuric acid. To control the evolution of foam, it is possible to use antifoams such as, for example, fatty acid polyglycol esters. To maintain the stability of plasmids, it is possible to add to the medium suitable selective substances, for example antibiotics. To maintain aerobic conditions, oxygen or oxygenous gas mixtures such as, for example, air are introduced into the culture.

The temperature of the culture is normally from 25° C. to 45° C. and preferably from 30° C. to 40° C. The activity of the microorganisms leads to an enrichment or accumulation of L-tryptophan in the fermentation or culture broth. The culture is continued until a maximum of L-tryptophan has formed. This target is normally reached within 10 hours to 160 hours. In continuous processes, longer cultivation times are possible.

A fermentation broth or culture broth is understood to mean a fermentation medium in which a microorganism has been cultivated for a certain time and at a certain temperature. On completion of the fermentation, the resulting fermentation broth accordingly comprises a) the biomass (=cell mass) of the microorganism that arises as a result of the propagation of the cells of the microorganism, b) the L-amino acid (L-tryptophan) formed during the fermentation, c) the organic by-products formed during the fermentation and d) the constituents of the fermentation medium/media used or of the starting materials that are not consumed by the fermentation, such as, for example, vitamins like thiamine or salts like magnesium sulphate.

The culture broth or fermentation broth produced can then be collected and the L-tryptophan or the L-tryptophan-containing product can be obtained or isolated. In one method variant, the fermentation broth is concentrated if necessary and then the L-tryptophan is purified or is isolated in a pure or virtually pure form. Typical methods for purifying L-amino acids such as L-tryptophan are ion-exchange chromatography, crystallization, extraction methods and treatment with activated carbon. What are obtained as a result are largely pure L-tryptophan having a content of ≥90% by weight, ≥95% by weight, ≥96% by weight, ≥97% by weight, ≥98% by weight or ≥99% by weight.

In another method variant, it is likewise possible to produce a product from the fermentation broth produced, by removing the bacterium biomass present in the fermentation broth to a complete extent (100%) or to a virtually complete extent, i.e. more than or greater than (>) 90%, >95%, >97%, >99%, and by leaving in the product the remaining constituents of the fermentation broth to a large extent, i.e. to an extent of 30%-100%, 40%-100%, 50%-100%, 60%-100%, 70%-100%, 80%-100%, or 90%-100%, preferably greater than or equal to (≥) 50%, ≥60%, ≥70%, ≥80%, ≥90% or ≥95%, or else to a complete extent (100%).

The biomass is removed or separated off by using separation methods such as, for example, centrifugation, filtration, decanting, flocculation or a combination thereof. The broth obtained is then thickened or concentrated using known methods such as, for example, with the aid of a rotary evaporator, thin-film evaporator, falling-film evaporator, by reverse osmosis, by nanofiltration or a combination thereof.

This concentrated broth is then worked up by methods of freeze drying, of spray drying, of spray granulation or by other processes to give a preferably free-flowing, fine powder. This free-flowing, fine powder can then in turn be converted by suitable compaction or granulation processes into a coarse, highly free-flowing, storable and substantially dust-free product.

Here, the water is altogether removed to an extent of more than 90%, and so the water content in the product is less than 10% by weight, less than 5% by weight, less than 4% by weight or less than 3% by weight.

The analysis of L-tryptophan to determine the concentration at one or more times during the fermentation can be effected by separating the L-tryptophan by means of ion-exchange chromatography, preferably cation-exchange chromatography, with subsequent post-column derivatization using ninhydrin, as described in Spackman et al. (Analytical Chemistry 30: 1190-1206 (1958)). It is also possible to employ ortho-phthalaldehyde rather than ninhydrin for post-column derivatization. An overview article on ion-exchange chromatography can be found in Pickering (LCGC (Magazine of Chromatographic Science) 7(6), 484-487 (1989)).

It is likewise possible to carry out a pre-column derivatization, for example using ortho-phthalaldehyde or phenyl isothiocyanate, and to fractionate the resulting amino acid derivatives by reversed-phase chromatography (RP), preferably in the form of high-performance liquid chromatography (HPLC). Such a method is, for example, described in Lindroth et al. (Analytical Chemistry 51: 1167-1174 (1979)). Detection is carried out photometrically (absorption, fluorescence).

The process and the microorganism according to the invention is used for fermentatively preparing L-tryptophan.

### BRIEF DESCRIPTION OF THE FIGURES

FIG. 1: Map of the DaroP deletion fragment-containing replacement vector pKO3DaroP

FIG. 2: Map of the mdfA gene-containing replacement vector pKO3DaroP::mdfA

FIG. 3: Map of the Dmtr deletion fragment-containing replacement vector pKO3Dmtr

FIG. 4: Map of the mdfA gene-containing replacement vector pKO3Dmtr::mdfA

FIG. 5: Map of the plasmid pMU91

Specified lengths are to be understood as approximations. The meanings of the abbreviations and designations used are as follows:
CmR: Chloramphenicol-resistance gene
sacB: sacB gene from Bacillus subtilis
RepA: Temperature-sensitive replication region of the plasmid pSC101
pdhR′: Part of the coding region of the pdhR gene
mdfA: Coding region of the mdfA gene
‘ampE: Part of the coding region of the ampE gene
‘yhbW: Part of the coding region of the yhbW gene
‘deaD: Part of the coding region of the deaD gene
pSC101: Plasmid fragment pSC101
serA: Coding region of the serA gene encoding D-3-phosphoglycerate dehydrogenase
trpE476DCBA: Part of the tryptophan operon trpLEDCBA with trpE476 allele
tetA: Tetracycline-resistance gene

The meanings of the abbreviations for the restriction enzymes are as follows
SaII: Restriction endonuclease from Streptomyces albus G
HindIII: Restriction endonuclease from Haemophilus influenzae Rd
XhoI: Restriction endonuclease from Xanthomonas holcicola

Further details may be found in the examples.

In the following, the invention is illustrated by non-limiting examples and exemplifying embodiments.

### EXAMPLES

Minimal (M9) and full media (LB) used for Escherichia coli are described by J. H. Miller (A Short Course in Bacterial Genetics (1992), Cold Spring Harbor Laboratory Press). The isolation of plasmid DNA from Escherichia coli and all techniques for restriction digestion, ligation, Klenow treatment and alkaline-phosphatase treatment are carried out as per Sambrook et al. (Molecular Cloning—A Laboratory Manual (1989) Cold Spring Harbor Laboratory Press). Unless otherwise described, the transformation of Escherichia coli is carried out in accordance with Chung et al. (Proceedings of the National Academy of Sciences of the United States of America, USA (1989) 86: 2172-2175).

Unless otherwise described, the incubation temperature in the production of strains and transformants is 37° C.

### Example 1

Construction of the Replacement Vector pKO3DaroP

The deletion flanks for the deletion of aroP are amplified using the polymerase chain reaction (PCR) and the deletion fragment was produced by overlap PCR. Proceeding from the nucleotide sequence of the aroP gene in E. coli K-12 MG1655 (accession number NC_000913.3, region: 120178-121551, Blattner et al. (Science 277: 1453-1462 (1997)) and the flanking regions, PCR primers are synthesized (Eurofins Genomics GmbH (Ebersberg, Germany)). The primers are designed such that the entire aroP gene is deleted.

Primer Design and PCR of the Two Flanks

```
Flank 1
aroP-up1
SEQ ID NO.: 3
5′ GATCTGAGCTCTAGACCTGGCGACGAAGCAACAAG 3′
                          Xbal
aroP-up6
SEQ ID NO.: 4
5′ GCGTTGGTGTAATCGCGAACCTCGTGCGGTGGTTGTT 3′
                                Nrul
```

The chromosomal E. coli MG1655 DNA used for the PCR is isolated using the QIAGEN DNeasy Blood & Tissue Kit (QIAGEN GmbH, Hilden, Germany) according to the information from the manufacturer. Using the two specific primers “aroP-up1” and “aroP-up6”, PCR amplification is carried out under standard PCR conditions (Innis et al.: PCR Protocols. A Guide to Methods and Applications, 1990, Academic Press) using the Phusion DNA Polymerase (Thermo Fisher Scientific, Wattham, Mass. USA) with MG1655 DNA as template to amplify the fragment “aroP-Flank1” (length: 775 bp). Via the primers, an XbaI and an NruI restriction site are inserted into the PCR product.

```
Flank 2
SEQ ID NO.: 5
aroP-down2 5′ GATCTATCTAGACTGTTACGCGCATTGCAGG 3′
                                          Xbal
aroP-down3
SEQ ID NO.: 6
5′ ACCGCACGAGGTTCGCGATTACACCAACGCCCCGTAAATCG 3′
                                Nrul
```

Using the two primers “aroP-down2” and “aroP-down3”, PCR is carried out with MG1655 DNA as template to amplify the fragment “aroP-Flank2” (length: 717 bp). Via the primers, an XbaI and an NruI restriction site are inserted into the PCR product.

Fusion of the Two Flanks by Overlap PCR

The two flanks are fused together via their overlapping regions by means of an overlap PCR using the two outer primers “aroP-up1” and “aroP-down2”. The resulting product “aroP-Del-Fragment” has a length of 1462 bp. Recognition sequences for the restriction enzyme XbaI are at both ends.

Cloning of the Insert into pKO3

The above fusion product is purified using the Qiaquick PCR Purification Kit (QIAGEN GmbH, Hilden, Germany) and then digested using XbaI. This generates XbaI “sticky ends”. The digest is purified again using the Qiaquick PCR Purification Kit, and this also results in the short cut-off sequences being removed.

The vector pKO3 is likewise cut using XbaI and simultaneously dephosphorylated using alkaline phosphatase. Thereafter, the digest is purified using the Qiaquick PCR Purification Kit, and this also results in the short fragment between the XbaI sites being removed. The restriction product likewise has XbaI “sticky ends”. These are unphosphorylated, preventing a self-ligation of the plasmid.

Ligation is carried out by using T4 ligase to ligate vector and insert in the molar ratio of 1:3. Chemically competent cells of the E. coli cloning strain NEB5alpha are transformed using 1 μl of the ligation reaction and spread out on LB agar containing 20 mg/l chloramphenicol. The plates are incubated at 30° C. for 40 h.

Checking of the Plasmid Clones

Successful cloning is verified by cleavage of the plasmid pKO3DaroP using the restriction enzyme AvaI.

10 colonies are picked and cultivated overnight at 30° C./180 rpm in 10 ml LB+20 mg/l chloramphenicol in each case.

The following day, 2 ml of the cultures are centrifuged in each case and minipreps are prepared from the pellets.

The ligation products can contain the insert in two orientations. An AvaI restriction allows to prove the insert as well as to determine the orientation:
Insert in orientation s: Fragments 148 bp, 1447 bp and 5494 bp
Insert in orientation as: Fragments 1351 bp, 1447 bp and 4291 bp
pKO3 empty vector: Fragments 1043 bp and 5263 bp

The 10 plasmid clones are cut using AvaI and the products are separated on a 0.8% TAE agarose gel. One plasmid clone containing the insert in orientation “s” was selected and called “pKO3DaroP”.

The insert of said clone is sequenced using the primers “pKO3-L” and “pKO3-R”.

SEQ ID NO.: 7
pKO3-L 5′ AGGGCAGGGTCGTTAAATAGC 3′
SEQ ID NO.: 8
pKO3-R 5′ TTAATGCGCCGCTACAGGGCG 3′

The DNA sequence of the amplified fragment “DaroP” is determined by using the primers “pKO3-L” and “pKO3-R” (Eurofins Genomics GmbH (Ebersberg, Germany)). The expected sequence of the fragment was confirmed and the cloned fragment is described in SEQ ID NO.:9.

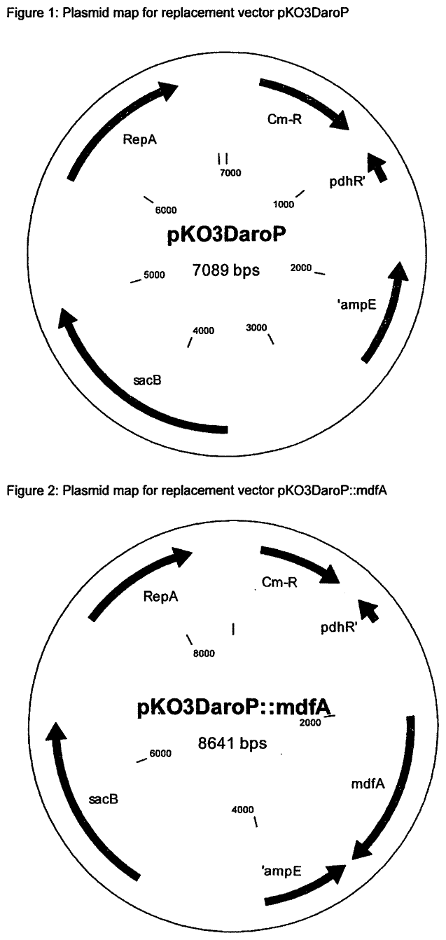

The resultant replacement vector pKO3DaroP is depicted in FIG. 1.

### Example 2

Construction of the Replacement Vector pKO3Dmtr

The deletion flanks for the deletion of mtr are amplified using the polymerase chain reaction (PCR) and the deletion fragment was produced by overlap PCR. Proceeding from the nucleotide sequence of the mtr gene in E. coli K-12 MG1655 (accession number NC_000913.3, region: 3304573-3305817, Blattner et al. (Science 277: 1453-1462 (1997)) and the flanking regions, PCR primers are synthesized (Eurofins Genomics GmbH (Ebersberg, Germany)). The primers are designed such that the entire mtr gene is deleted.

Primer Design and PCR of the Two Flanks

Flank 1
SEQ ID NO.: 10
mtr-del-Xba-1 5′ TTGAGAACCGCGAGCGTCGTCTG 3′
mtr-del-Xba-2
SEQ ID NO.: 11
5′ TCTAGATTCGCGAGGGCTTCTCTCCAGTGAAA 3′
Xbal Nrul

The chromosomal E. coli MG1655 DNA used for the PCR is isolated using the QIAGEN DNeasy Blood & Tissue Kit (QIAGEN GmbH, Hilden, Germany) according to the information from the manufacturer. Using the two specific primers “mtr-del-Xba-1” and “mtr-del-Xba-2”, PCR is carried out under standard PCR conditions (Innis et al.: PCR Protocols. A Guide to Methods and Applications, 1990, Academic Press) using the Phusion DNA Polymerase (Thermo Fisher Scientific, Wattham, Mass. USA) with MG1655 DNA as template to amplify the fragment “mtr-Flank1” (length: 1009 bp). Via the primers, an XbaI and an NruI restriction site are inserted into the PCR product.

Flank 2
mtr-del-Xba-3
SEQ ID NO.: 12
5′ AAGCCCTCGCGAATCTAGAGTAATCAGCGGTGCCTTATC 3′
Nrul Xbal
SEQ ID NO.: 13
mtr-del-Xba-4 5′ GCTGGCAGAACACCACAATATG 3′

Using the two primers “mtr-del-Xba-3” and “mtr-del-Xba-4”, PCR is carried out with MG1655 DNA as template to amplify the fragment “mtr-Flank2” (length: 1014 bp). Via the primers, an XbaI and an NruI restriction site are inserted into the PCR product.

Fusion of the Two Flanks by Overlap PCR

The two flanks are fused together via their overlapping regions by means of an overlap PCR using the two outer primers “mtr-del-Xba-1” and “mtr-del-Xba-4”. The resulting product “mtr-del-Fragment” has a length of 2004 bp.

Cloning of the Insert into pKO3

According to information from the manufacturer, the amplified “mtr-del-Fragment” is ligated into the vector pCR-Blunt II-TOPO (Zero Blunt TOPO PCR Cloning Kit, Thermo Fisher Scientific, Wattham, Mass. USA) and transformed into E. coli TOP10. Cells containing a plasmid are selected on LB agar containing 50 μg/ml kanamycin. After isolation of the plasmid DNA, successful cloning is verified by cleavage of the plasmid using the restriction enzyme HincII and separation of the products on a 0.8% TAE agarose gel. The successfully cloned vector is called “pCRBI-mtr-del”.

The vector is then cleaved using the restriction enzymes NotI and SpeI and the mtr-del-Fragment is resolved on a 0.8% TAE agarose gel. This is followed by the isolation of the fragment from the gel using the QIAquick Gel Extraction Kit (QIAGEN GmbH, Hilden, Germany) and cloning into the replacement vector pKO3 (Link et al, 1997, J. Bacteriol., 179, 20, 6228-6237).

The vector pKO3 is cut using NotI and XbaI. Thereafter, the digest is purified using the Qiaquick PCR Purification Kit (QIAGEN GmbH, Hilden, Germany).

Ligation is carried out by using T4 ligase to ligate vector and insert in the molar ratio of 1:3. Chemically competent cells of the E. coli cloning strain NEB5alpha are transformed using 1 μl of the ligation reaction and spread out on LB agar containing 20 mg/l chloramphenicol. The plates are incubated at 30° C. for 40 h.

Checking of the Plasmid Clones

Successful cloning is verified by cleavage of the plasmid pKO3Dmtr using the restriction enzyme HincII.

Correct clones have the following cleavage pattern:
Insert mtr-del: Fragments 822 bp, 1251 bp, 1611 bp and 4020 bp
pKO3 empty vector: Fragments 822 bp, 1617 bp and 3232 bp

10 colonies are picked and cultivated overnight at 30° C./180 rpm in 10 ml LB+20 mg/l chloramphenicol in each case.

The following day, 2 ml of the cultures are centrifuged in each case and minipreps are prepared from the pellets.

The 10 plasmid clones are cut using HincII and the products are separated on a 0.8% TAE agarose gel. One plasmid clone containing the correct insert was selected and called “pKO3Dmtr”.

The insert of said clone is sequenced using the primers “pKO3-L” and “pKO3-R”.

SEQ ID NO.: 7
pKO3-L 5′ AGGGCAGGGTCGTTAAATAGC 3′
SEQ ID NO.: 8
pKO3-R 5′ TTAATGCGCCGCTACAGGGCG 3′

The DNA sequence of the amplified fragment “Dmtr” is determined by using the primers “pKO3-L” and “pKO3-R” (Eurofins Genomics GmbH (Ebersberg, Germany)). The expected sequence of the fragment was confirmed and the cloned fragment is described in SEQ ID NO.:14.

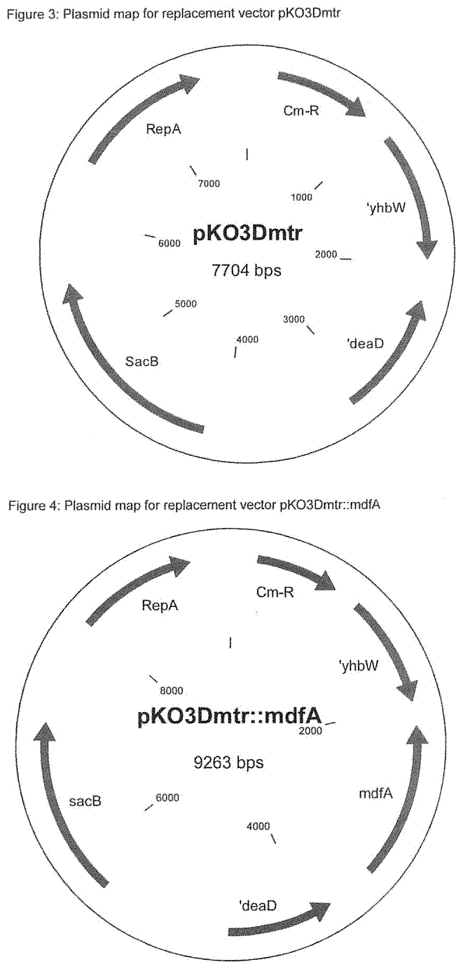

The resultant replacement vector pKO3Dmtr is depicted in FIG. 3.

### Example 3

Construction of the Replacement Vectors pKO3DaroP::mdfA and pKO3Dmtr::mdfA

3.1 Construction of the Vector pB-mdfA

The mdfA allele is amplified using the polymerase chain reaction (PCR). Proceeding from the nucleotide sequence of the mdfA gene in E. coli K12 MG1655 (accession number NC_000913.3, region: 883673-884905, Blattner et al. (Science 277: 1453-1462 (1997)), PCR primers are synthesized (Eurofins Genomics GmbH (Ebersberg, Germany)).

Primer Design and PCR of the mdfA Gene Region

```
SEQ ID NO.: 15
cmr-for: 5′ CGTCGCGATACAGGCAAGTCGTTGAG 3′
                              Nrul
SEQ ID NO.: 16
cmr-rev: 5′ CCTCGCGACAGATTGACGACCATCAC 3′
                              Nrul
```

The chromosomal E. coli MG1655 DNA used for the PCR is isolated using the QIAGEN DNeasy Blood & Tissue Kit (QIAGEN GmbH, Hilden, Germany) according to the information from the manufacturer. Using the two specific primers “crmr-for” and “cmr-rev”, PCR is carried out under standard PCR conditions (Innis et al.: PCR Protocols. A Guide to Methods and Applications, 1990, Academic Press) using the Phusion DNA Polymerase (Thermo Fisher Scientific, Wattham, Mass. USA) with MG1655 DNA as template to amplify the fragment “mdfA-Insert” (length: 1562 bp), described in SEQ ID NO.: 19. Via the primers, NruI restriction sites are inserted into the PCR product.

Cloning of the Insert into pCR-Blunt II

According to information from the manufacturer, the amplified fragment “mdfA-Insert” is ligated into the vector pCR-Blunt II-TOPO (Zero Blunt TOPO PCR Cloning Kit, Thermo Fisher Scientific, Wattham, Mass. USA) and transformed into E. coli TOP10. Cells containing a plasmid are selected on LB agar containing 50 μg/ml kanamycin. After isolation of the plasmid DNA, successful cloning is verified by cleavage of the plasmid using the restriction enzyme SphI and separation of the products on a 0.8% TAE agarose gel.

The ligation products can contain the insert in two orientations. A SphI restriction allows to prove the insert as well as to determine the orientation:
Insert in orientation s: Fragments 1351 bp, 1385 bp and 2345 bp
Insert in orientation as: Fragments 305 bp, 1385 bp and 3391 bp
pCR-Blunt II empty vector: Fragments 1385 bp and 2134 bp

The plasmid clones are cut using SphI and the products are separated on a 0.8% TAE agarose gel. One plasmid clone containing the insert in orientation “as” was selected and called “pB-mdfA”.

The insert of said clone is sequenced using the primers “M13uni(-21)” and “M13rev(-29)”.

SEQ ID NO.: 17
M13uni(-21) 5′ TGTAAAACGACGGCCAGT 3′
SEQ ID NO.: 18
M13rev(-29) 5′ CAGGAAACAGCTATGACC 3′

The DNA sequence of the amplified fragment “mdfA” is determined by using the primers “M13uni(-21)” and “M13rev(-29)” (Eurofins Genomics GmbH (Ebersberg, Germany)). The expected sequence of the fragment was confirmed.

3.2 Construction of the Vector pKO3DaroP::mdfA

The vector pB-mdfA is cleaved using the restriction enzyme NruI and the mdfA fragment is resolved on a 0.8% TAE agarose gel. This is followed by the isolation of the fragment from the gel using the QIAquick Gel Extraction Kit (QIAGEN GmbH, Hilden, Germany) and cloning into the replacement vector pKO3DaroP that was generated.

The vector pKO3DaroP is cut using NruI and simultaneously dephosphorylated using alkaline phosphatase. This cleavage preserves the promoter region of aroP that is still present. Thereafter, the digest is purified using the Qiaquick PCR Purification Kit (QIAGEN GmbH, Hilden, Germany).

Ligation is carried out by using T4 ligase to ligate vector and insert in the molar ratio of 1:3. Chemically competent cells of the E. coli cloning strain NEB5alpha are transformed using 1 μl of the ligation reaction and spread out on LB agar containing 20 mg/l chloramphenicol. The plates are incubated at 30° C. for 40 h.

Checking of the Plasmid Clones

Successful cloning is verified by cleavage of the plasmid pKO3DaroP::mdfA using the restriction enzyme AseI.

10 colonies are picked and cultivated overnight at 30° C./180 rpm in 10 ml LB+20 mg/l chloramphenicol in each case.

The following day, 2 ml of the cultures are centrifuged in each case and minipreps are prepared from the pellets.

The ligation products can contain the insert in two orientations. An AseI restriction allows to prove the insert as well as to determine the orientation:
Insert in orientation s: Fragments 1603 bp and 7038 bp
Insert in orientation as: Fragments 101 bp and 8540 bp

The 10 plasmid clones are cut using AseI and the products are separated on a 0.8% TAE agarose gel. One plasmid clone containing the insert in orientation “s” was selected and called “pKO3DaroP::mdfA”.

The insert of said clone is sequenced using the primers “pKO3-L”, “pKO3-R”, “cmr-for” and “cmr-rev”.

SEQ ID NO.: 7
pKO3-L 5′ AGGGCAGGGTCGTTAAATAGC 3′
SEQ ID NO.: 8
pKO3-R 5′ TTAATGCGCCGCTACAGGGCG 3′
SEQ ID NO.: 15
cmr-for 5′ TACAGGCAAGTCGTTGAG 3′
SEQ ID NO.: 16
cmr-rev 5′ CAGATTGACGACCATCAC 3′

The DNA sequence of the insert “DaroP::mdfA” is determined by using the primers “pKO3-L”, “pKO3-R”, “cmr-for” and “cmr-rev” (Eurofins Genomics GmbH (Ebersberg, Germany)).

The expected sequence of the fragment was confirmed and the cloned fragment is described in SEQ ID NO.: 20.

The resultant replacement vector pKO3DaroP::mdfA is depicted in FIG. 2.

3.3 Construction of the Vector pKO3Dmtr::mdfA

The vector pB-mdfA is cleaved using the restriction enzyme NruI and the mdfA fragment is resolved on a 0.8% TAE agarose gel. This is followed by the isolation of the fragment from the gel using the QIAquick Gel Extraction Kit (QIAGEN GmbH, Hilden, Germany) and cloning into the replacement vector pKO3Dmtr that was generated.

The vector pKO3Dmtr is cut using NruI and simultaneously dephosphorylated using alkaline phosphatase. This cleavage preserves the promoter region of mtr that is still present. Thereafter, the digest is purified using the Qiaquick PCR Purification Kit (QIAGEN GmbH, Hilden, Germany).

Ligation is carried out by using T4 ligase to ligate vector and insert in the molar ratio of 1:3. Chemically competent cells of the E. coli cloning strain NEB5alpha are transformed using 1 μl of the ligation reaction and spread out on LB agar containing 20 mg/l chloramphenicol. The plates are incubated at 30° C. for 40 h.

Checking of the Plasmid Clones

Successful cloning is verified by cleavage of the plasmid pKO3Dmtr::mdfA using the restriction enzyme HincII.

10 colonies are picked and cultivated overnight at 30° C./180 rpm in 10 ml LB+20 mg/l chloramphenicol in each case.

The following day, 2 ml of the cultures are centrifuged in each case and minipreps are prepared from the pellets.

The ligation products can contain the insert in two orientations. A HincII restriction allows to prove the insert as well as to determine the orientation:
Insert in orientation s: Fragments 354 bp, 822 bp, 1613 bp, 2449 bp and 4025 bp
Insert in orientation as: Fragments 822 bp, 1181 bp, 1613 bp, 1622 bp and 4025 bp

The 10 plasmid clones are cut using HincII and the products are separated on a 0.8% TAE agarose gel. One plasmid clone containing the insert in orientation “s” was selected and called “pKO3Dmtr::mdfA”.

The insert of said clone is sequenced using the primers “pKO3-L”, “pKO3-R”, “cmr-for” and “cmr-rev”.

SEQ ID NO.: 7
pKO3-L 5′ AGGGCAGGGTCGTTAAATAGC 3′
SEQ ID NO.: 8
pKO3-R 5′ TTAATGCGCCGCTACAGGGCG 3′
SEQ ID NO.: 15
cmr-for 5′ TACAGGCAAGTCGTTGAG 3′
SEQ ID NO.: 16
cmr-rev 5′ CAGATTGACGACCATCAC 3′

The DNA sequence of the insert “Dmtr::mdfA” is determined by using the primers “pKO3-L”, “pKO3-R”, “cmr-for” and “cmr-rev” (Eurofins Genomics GmbH (Ebersberg, Germany)). The expected sequence of the fragment was confirmed and the cloned fragment is described in SEQ ID NO.: 21.

The resultant replacement vector pKO3Dmtr::mdfA is depicted in FIG. 4.

### Example 4

Replacement of the aroP Allele of Strain DM2186 by the Deletion Fragments DaroP and DaroP::mdfA

The L-tryptophan-producing E. coli strain DM2186/pMU91 is a P1-transduction-obtainable trpS⁺ derivative of the Escherichia coli K-12 strain JP6015/pMU91 described in the patent specification U.S. Pat. No. 5,756,345. pMU91 is a plasmid derived from pSC101 (Cohen et al., Journal of Bacteriology 132: 734-737 (1977)), which bears Tet^{R}, trpE476DCBA and serA⁺. JP6015/pMU91 is deposited as DSM 10123 at the Leibniz Institute DSMZ—German Collection of Microorganisms and Cell Cultures (DSMZ, Braunschweig, Germany) in accordance with the Budapest treaty. DM2186 is deposited on Nov. 22, 2018 as DSM 32961 at the Leibniz Institute DSMZ—German Collection of Microorganisms and Cell Cultures (DSMZ, Braunschweig, Germany) in accordance with the Budapest treaty.

In the chromosomal aroP allele of the strain DM2186, there is a frame-shift mutation.

The replacement of the chromosomal aroP allele by the plasmid-coded deletion construct was carried out by transforming DM2186 with the plasmid pKO3DaroP. The gene replacement is done using the selection method described by Link et al. (Journal of Bacteriology 179: 6228-6237 (1997)) and was verified by sequencing.

After replacement has been carried out, what is present in DM2186 is the form of the deleted aroP allele that is described in SEQ ID NO.:22 (sequencing by Eurofins Genomics GmbH (Ebersberg, Germany)). The strain obtained is referred to as DM2186DaroP.

Increased Expression of the mdfA Gene in the E. coli Strain DM2186/pMU91 Integrated in the aroP Locus

The replacement of the chromosomal aroP allele by the plasmid-coded mdfA allele with simultaneous deletion of the aroP gene, the promoter region of aroP being preserved, was carried out by transforming DM2186 with the plasmid pKO3DaroP::mdfA. The gene replacement is done using the selection method described by Link et al. (Journal of Bacteriology 179: 6228-6237 (1997)) and was verified by sequencing.

After replacement has been carried out, what is present in DM2186 is the form of the mdfA gene, as described in SEQ ID NO.: 23, integrated in the aroP locus with simultaneous deletion of the aroP allele (sequencing by Eurofins Genomics GmbH (Ebersberg, Germany)). The strain obtained is referred to as DM2186DaroP::mdfA.

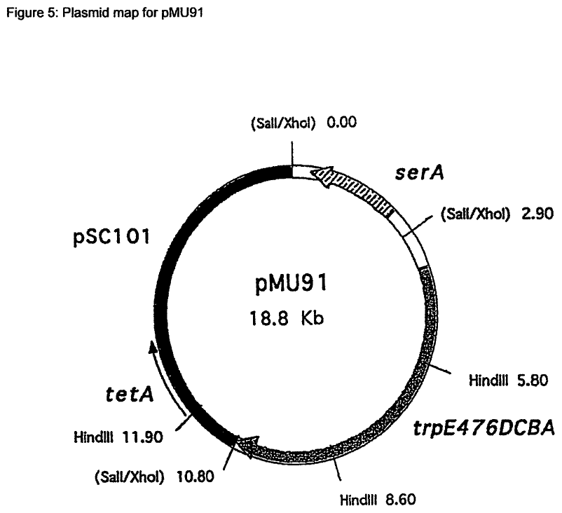

The plasmid pMU91 which is described in the patent specification U.S. Pat. No. 5,756,345 and which carries the genetic information for tryptophan production was isolated from the strain JP6015/pMU91. The plasmid is depicted in FIG. 5. The strains DM2186DaroP and DM2186DaroP::mdfA were each transformed with this plasmid. The respective transformants are referred to as DM2186DaroP/pMU91 and DM2186DaroP::mdfA/pMU91.

### Example 5

Replacement of the Mtr Allele of Strain DM2186 by the Deletion Fragments Dmtr and Dmtr::mdfA

The L-tryptophan-producing E. coli strain DM2186/pMU91 is a P1-transduction-obtainable trpS⁺ derivative of the Escherichia coli K-12 strain JP6015/pMU91 described in the patent specification U.S. Pat. No. 5,756,345. pMU91 is a plasmid derived from pSC101 (Cohen et al., Journal of Bacteriology 132: 734-737 (1977)), which bears Tet^{R}, trpE476DCBA and serA⁺. JP6015/pMU91 is deposited as DSM 10123 at the Leibniz Institute DSMZ—German Collection of Microorganisms and Cell Cultures (DSMZ, Braunschweig, Germany) in accordance with the Budapest treaty. DM2186 is deposited on Nov. 22, 2018 as DSM 32961 at the Leibniz Institute DSMZ—German Collection of Microorganisms and Cell Cultures (DSMZ, Braunschweig, Germany) in accordance with the Budapest treaty.

In the chromosomal mtr allele of the strain DM2186, there is a frame-shift mutation.

The replacement of the chromosomal mtr allele by the plasmid-coded deletion construct was carried out by transforming DM2186 with the plasmid pKO3Dmtr. The gene replacement is done using the selection method described by Link et al. (Journal of Bacteriology 179: 6228-6237 (1997)) and was verified by sequencing.

After replacement has been carried out, what is present in DM2186 is the form of the deleted mtr allele that is described in SEQ ID NO.: 24 (sequencing by Eurofins Genomics GmbH (Ebersberg, Germany)). The strain obtained is referred to as DM2186Dmtr.

Increased Expression of the mdfA Gene in the E. coli Strain DM2186/pMU91 Integrated in the Mtr Locus

The replacement of the chromosomal mtr allele by the plasmid-coded mdfA allele with simultaneous deletion of the mtr gene, the promoter region of mtr being preserved, was carried out by transforming DM2186 with the plasmid pKO3Dmtr::mdfA. The gene replacement is done using the selection method described by Link et al. (Journal of Bacteriology 179: 6228-6237 (1997)) and was verified by sequencing.

After replacement has been carried out, what is present in DM2186 is the form of the mdfA gene, as described in SEQ ID NO.: 25, integrated in the mtr locus with simultaneous deletion of the mtr allele (sequencing by Eurofins Genomics GmbH (Ebersberg, Germany)). The strain obtained is referred to as DM2186Dmtr::mdfA.

The plasmid pMU91 which is described in the patent specification U.S. Pat. No. 5,756,345 and which carries the genetic information for tryptophan production was isolated from the strain JP6015/pMU91. The plasmid is depicted in FIG. 5. The strains DM2186Dmtr and DM2186Dmtr::mdfA were each transformed with this plasmid. The respective transformants are referred to as DM2186Dmtr/pMU91 and DM2186Dmtr::mdfA/pMU91.

### Example 6

Production of L-Tryptophan Using the Strains DM2186DaroP/pMU91, DM2186DaroP::mdfA/pMU91, DM2186Dmtr/pMU91 and DM2186Dmtr::mdfA/pMU91

To test the effect of an additional copy of the mdfA gene expressed from various regulatory sequences in two different chromosomal environments DM2186DaroP/pMU91, DM2186DaroP::mdfA/pMU91, DM2186Dmtr/pMU91, DM2186Dmtr::mdfA/pMU91 and DM2186/pMU91 were further propagated at 30° C. on LB medium having the following composition: 10 g/l Bacto tryptone, 5 g/l yeast extract, 10 g/l NaCl (pH adjustment to 7.5 using NaOH), 2 g/l glucose, 20 g/l agar and 5 mg/l tetracycline. The formation of L-tryptophan is checked in batch cultures of 10 ml contained in 100 ml conical flasks. To this end, 10 ml of preculture medium of the following composition are inoculated, and incubated at 30° C. and 180 rpm for 16 hours on an Infors HT Multitron standard incubator from Infors AG (Bottmingen, Switzerland): 1 g/l yeast extract, 100 ml/I MOPS buffer (10×), 10 g/l glucose, 0.1 mg/l thiamine and 5 mg/l tetracycline.

10×MOPS buffer is prepared from solutions A and B according to Tables 1 to 3; two volumes of solution A are added aseptically to three volumes of solution B.

TABLE 1
Solution A for 10x MOPS buffer (sterile-filtered).

| Component | Concentration |
|---|---|
| MOPS (morpholinopropanesulfonic acid) | 418.6 g/L |
| KOH (solid) | For adjustment of |
| | pH to 7.4 |

TABLE 2
Solution B for 10x MOPS buffer. Sterilized
by autoclaving (30 minutes, 121° C.).

| Component | Concentration | |
|---|---|---|
| Na₃ citrate × 2H₂O | 2.35 | g/l |
| FeSO₄ × 7H₂O | 0.22 | g/l |
| NH₄Cl | 32.0 | g/l |
| MgSO₄ × 7H₂O | 6.7 | g/l |
| KCl | 4.0 | g/l |
| CaCl₂ × 2H₂O | 0.25 | mg/l |
| Trace-elements stock solution (Tab. 4) | 3.33 | ml/l |

TABLE 3
Trace-elements stock solution (in
demin. water) for 10x MOPS buffer.

| Component | Concentration | |
|---|---|---|
| (NH₄)₆Mo₇O₂₄ × 4H₂O | 3.7 | mg/l |
| H₃BO₃ | 24.0 | mg/l |
| CoCl₂ × 6H₂O | 7.1 | mg/l |
| ZnSO₄ × 7H₂O | 28.7 | mg/l |
| MnCl₂ × 4H₂O | 15.8 | mg/l |
| CuSO₄ × 5H₂O | 2.5 | mg/l |

100 μl of said preculture are in each case inoculated in 10 ml of production medium PM3P according to Table 4 and incubated at 33° C. and 180 rpm for 44 hours on an Infors HT Multitron standard incubator from Infors AG (Bottmingen, Switzerland). After incubation, the optical density (OD) of the culture suspension is determined using a Nanocolor 400D photometer from Macherey-Nagel GmbH & Co. KG (Diren, Germany) at a measurement wavelength of 660 nm.

TABLE 4
PM3P medium: for production tests in 100 ml conical flasks

| Component | Concentration | |
|---|---|---|
| 10x MOPS buffer | 100 | ml/l |
| Thiamine HCl (0.01%) | 1.0 | ml/l |
| KH2PO4 (45 mM) | 10 | ml/l |
| Glucose | 10 | g/l |
| Tyrosine | 0.02 | g/l |
| Phenylalanine | 0.02 | g/l |
| Tetracycline | 5 | mg/l |

Thereafter, the concentration of L-tryptophan formed in the sterile-filtered culture supernatant is determined by reversed-phase HPLC, for instance as described in Lindroth et al. (Analytical Chemistry (1979) 51: 1167-1174).

Table 5 shows the result of the experiment.

TABLE 5
Production of L-tryptophan using the strains
DM2186DaroP/pMU91, DM2186DaroP::mdfA/pMU91,
DM2186Dmtr/pMU91 and DM2186Dmtr::mdfA/pMU91

| | OD | L-Tryptophan, | Yield, |
|---|---|---|---|
| Strain | (660 nm) | g/l | net % |
| DM2186DaroP/pMU91 | 4.17 | 1.63 | 13.60 |
| DM2186DaroP::mdfA/pMU91 | 4.02 | 1.66 | 13.82 |
| DM2186Dmtr/pMU91 | 4.29 | 1.62 | 13.61 |
| DM2186Dmtr::mdfA/pMU91 | 3.85 | 1.82 | 15.16 |
| DM2186/pMU91 | 4.38 | 1.62 | 13.54 |

As derivable therefrom, the enhancement of mdfA gene increases the yield of L-tryptophan. However, the expression level of mdfA—and thereby the L-tryptophan yield—is strongly influenced by choosing the integration locus and the chromosomal environment of the integrated mdfA gene or allele.

Integration of the mdfA into the chromosomal aroP locus leads to a slight increase in L-tryptophan productivity, whereas integration of the mdfA into the chromosomal mtr locus leads to a strong increase in L-tryptophan productivity, i.e. the chromosomal environment regulates the expression of mdfA. The positive effect on L-tryptophan synthesis is much more pronounced in DM2186Dmtr::mdfA/pMU91 compared to the integration of mdfA into the aroP gene locus. Obviously, creation of a defined expression level of the additional copy of the mdfA gene in a specific gene locus is more important for L-tryptophan production than the general enhancement of expression by gene copy number increasement. Especially the expression levels of membrane proteins, such as transport proteins and amino acid exporters have to be modulated in an appropriate way without affecting cellular fitness and production capacities when commercial relevant concentrations accumulate in the fermentation broth.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

1-15. (canceled)

16. A method of producing L-tryptophan, comprising culturing an L-tryptophan producing microorganism belonging to the Enterobacteriaceae family in a fermentation medium, wherein L-tryptophan producing microorganism has been modified by enhancing the expression level of an mdfA gene or by enhancing the expression level of an mdfA allele.

17. The method of claim 16, wherein the mdfA allele is a polynucleotide selected from the group consisting of:
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2; and
g) a polynucleotide encoding a protein comprising an amino acid sequence which includes substitution, deletion, insertion or addition of 1 to 20 amino acids in SEQ ID NO.:2.

18. The method of claim 16, wherein the expression level of the mdfA gene or of the mdfA allele is enhanced by increasing the copy number of the mdfA gene or of the mdfA allele.

19. The method of claim 18, wherein the copy number of the mdfA gene or of the mdfA allele is increased by integrating the gene into the chromosome of the microorganism.

20. The method of claim 16, wherein the expression level of the mdfA gene or of the mdfA allele is enhanced by modifying a regulatory sequence of the gene.

21. The method of claim 16, wherein the expression level of the mdfA gene or of the mdfA allele is enhanced by using an inducible promoter.

22. The method of claim 16, wherein a copy of the mdfA gene or of the mdfA allele is integrated in the mtr locus with simultaneous deletion of the mtr gene.

23. The method of claim 22, wherein the chromosomal environment of the mtr locus regulates the expression of the mdfA gene or the mdfA allele integrated in the mtr locus.

24. The method of claim 16, wherein the L-tryptophan producing microorganism is selected from the genera Escherichia, Erwina and Providencia.

25. The method of claim 16, wherein the L-tryptophan producing microorganism is Escherichia coli.

26. A method for enhancing the expression level of the mdfA gene or of an mdfA allele in a microorganism, wherein the enhanced expression is due to transformation, transduction or conjugation of the microorganism by a vector comprising any one of the following:
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2;
g) a polynucleotide encoding a protein comprising an amino acid sequence which includes substitution, deletion, insertion or addition of 1 to 20 amino acids in SEQ ID NO.:2;
and comprising a promoter regulating the expression of polynucleotides a) to g).

27. The method of claim 26, wherein said microorganism is of the Enterobacteriaceae family.

28. An L-tryptophan producing microorganism wherein said microorganism comprises a polynucleotide selected from the group consisting of:
a) a polynucleotide sequence comprising the nucleotide sequence of SEQ ID NO.:1;
b) a polynucleotide that hybridizes with a complementary strand of SEQ ID NO.:1 under stringent conditions;
c) a naturally occurring mutant or polymorphic form of a polynucleotide according to a) or b);
d) a polynucleotide having a sequence identity of at least 80% of the nucleotide sequence of SEQ ID NO.:1;
e) a polynucleotide including substitutions, deletions, insertions or additions of 1 to 60 nucleotides in relation to the nucleotide sequence of SEQ ID NO.:1;
f) a polynucleotide encoding a protein comprising the amino acid sequence of SEQ ID NO.:2; and
g) a polynucleotide encoding a protein comprising an amino acid sequence which includes substitution, deletion, insertion or addition of 1 to 20 amino acids in SEQ ID NO.:2.

29. The L-tryptophan producing microorganism of 28, wherein a copy of the mdfA gene or of the mdfA allele is integrated in the mtr locus of the microorganism in the place of the mtr gene.

30. The L-tryptophan producing microorganism of 29, wherein the chromosomal environment of the mtr locus regulates the expression of the mdfA gene or the mdfA allele integrated in the mtr locus


SEQUENCE LISTING (25 sequences)

SEQ ID NO: 1 - DNA, 1233 bp, Escherichia coli
  CDS (1)..(1233): Coding region mdfA
atgcaaaataaattagcttccggtgccaggcttggacgtcaggcgttacttttccctctctgtctggtgctttacgaattttcaacctatatcggcaacgatatgattcaacccggtatgttggccgtggtggaacaatatcaggcgggcattgattgggttcctacttcgatgaccgcgtatctggcgggcgggatgtttttacaatggctgctggggccgctgtcggatcgtattggtcgccgtccggtgatgctggcgggagtggtgtggtttatcgtcacctgtctggcaatattgctggcgcaaaacattgaacaattcaccctgttgcgcttcttgcagggcataagcctctgtttcattggcgctgtgggatacgccgcaattcaggaatccttcgaagaggcggtttgtatcaagatcaccgcgctgatggcgaacgtggcgctgattgctccgctacttggtccgctggtgggcgcggcgtggatccatgtgctgccctgggaggggatgtttgttttgtttgccgcattggcagcgatctcctttttcggtctgcaacgagccatgcctgaaaccgccacgcgtataggcgagaaactgtcactgaaagaactcggtcgtgactataagctggtgctgaagaacggccgctttgtggcgggggcgctggcgctgggattcgttagtctgccgttgctggcgtggatcgcccagtcgccgattatcatcattaccggcgagcagttgagcagctatgaatatggcttgctgcaagtgcctattttcggggcgttaattgcgggtaacttgctgttagcgcgtctgacctcgcgccgcaccgtacgttcgctgattattatgggcggctggccgattatgattggtctattggtcgctgctgcggcaacggttatctcatcgcacgcgtatttatggatgactgccgggttaagtatttatgctttcggtattggtctggcgaatgcgggactggtgcgattaaccctgtttgccagcgatatgagtaaaggtacggtttctgccgcgatgggaatgctgcaaatgctgatctttaccgttggtattgaaatcagcaaacatgcctggctgaacgggggcaacggactgtttaatctcttcaaccttgtcaacggaattttgtggctgtcgctgatggttatctttttaaaagataaacagatgggaaattctcacgaagggtaa

SEQ ID NO: 2 - protein, 410 aa, Escherichia coli
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNGRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHEG

SEQ ID NO: 3 - DNA, 35 bp, Escherichia coli
  misc_feature (1)..(35): Primer aroP-up1
gatctgagctctagacctggcgacgaagcaacaag

SEQ ID NO: 4 - DNA, 37 bp, Escherichia coli
  misc_feature (1)..(37): Primer aroP-up6
gcgttggtgtaatcgcgaacctcgtgcggtggttgtt

SEQ ID NO: 5 - DNA, 31 bp, Escherichia coli
  misc_feature (1)..(31): Primer aroP-down2
gatctatctagactgttacgcgcattgcagg

SEQ ID NO: 6 - DNA, 41 bp, Escherichia coli
  misc_feature (1)..(41): Primer aroP-down3
accgcacgaggttcgcgattacaccaacgccccgtaaatcg

SEQ ID NO: 7 - DNA, 21 bp, Artificial Sequence
  misc_feature (1)..(21): Primer pKO3-L
agggcagggtcgttaaatagc

SEQ ID NO: 8 - DNA, 21 bp, Artificial Sequence
  misc_feature (1)..(21): Primer pKO3-R
ttaatgcgccgctacagggcg

SEQ ID NO: 9 - DNA, 1568 bp, Escherichia coli
  misc_feature (1)..(1568): DaroP; construct for deletion of aroP
agggcagggtcgttaaatagccgcttatgtctattgctggtctcggtacccggggatcgcggccgcggaccggatcctctagacctggcgacgaagcaacaagcccttcgcttcgagacgttgaatcgcctcacgcaaggagggacgggagacgtcaaactgttttgccagttcgcgttccggtgggagtttttcgcccgggcggagagtgccttcgaggatcaaaaactccagttgctgctcaatcacatcggagagttttggttggcggattttgctgtaggccatgagttcctgtcttaagccacttgccgaagtcaattggtcttaccaatttcatgtctgtgacgctaaagtaacaaagtattcaccttatgtccatacaggttttgattgaaatcatgaaactgtgcacattttaacaacttgacatatataacgtttcaaagttgtaactatgcacaaatgtagactttacgtaggaaaggagttttttaacgattattaaactataaaaatccgaattgaaccgattcacttaccaattttgtgatttttaattcaattaaaacgaatttaaattcattctacatattgagaggggttgaggctgagctttacaaacggtttctttttaagcaactcatcttcaaccatgcataaagcgggtgcattcgctgccgcataccattattcttgatctgacggaagtctttttgtaacaattcaaacttctttgatgtaaacaaattaatacaacaaacggaattgcaaacttacacacgcatcactgcgtagatcaaaaaaacaaccaccgcacgaggttcgcgattacaccaacgccccgtaaatcgtcagtagtgcaatcaccaccacgaccacgaacgaggttttcttcgccattgaaaccgctgccttcggcgtctccaccttatcgacatgcggttcacgcgccagagagaactgcgccagacgcgttaacacctgatactgcgaagtatggaaatcacccagcgaagcaaaccaggccggtaacgctttctcaccatgaccgatcaaggcatataccacacccgcaagacgaaccggcacccaatccagtacatgaagcacggcatcaatgccggactgtaaacgatgatgcggcgtctgatatcgcgccagccagtattgccatgcacgcaaaaacgcataccccatcagcgtaacgggtccccaggttccccccacaatcagccagaacagcggtgcaagataaaaacgaaagttaatccacagcaatgcattttgcagctcacgcaaatactcacgttcgtcgcagcctgccgggacgccgtgaatcatggtgagttcgccagccatcgtggcacgggcatggctatcattacgtgaagcagctgtcagataagcatgataatgaagacgaactttacctgcgccaatacacagcaaaccaatcagcagccacaccagtagcgtgggaacgttgaacaatactccctgcaatgcgcgtaacagtctagagtcgaccggtggcgaatgggacgcgccctgtagcggcgcattaa

SEQ ID NO: 10 - DNA, 23 bp, Escherichia coli
  misc_feature (1)..(23): Primer mtr-del-Xba-1
ttgagaaccgcgagcgtcgtctg

SEQ ID NO: 11 - DNA, 32 bp, Escherichia coli
  misc_feature (1)..(32): Primer mtr-del-Xba-2
tctagattcgcgagggcttctctccagtgaaa

SEQ ID NO: 12 - DNA, 39 bp, Escherichia coli
  misc_feature (1)..(39): Primer mtr-del-Xba-3
aagccctcgcgaatctagagtaatcagcggtgccttatc

SEQ ID NO: 13 - DNA, 22 bp, Escherichia coli
  misc_feature (1)..(22): Primer mtr-del-Xba-4
gctggcagaacaccacaatatg

SEQ ID NO: 14 - DNA, 2183 bp, Escherichia coli
  misc_feature (1)..(2183): Dmtr; construct for deletion of mtr
agggcagggtcgttaaatagccgcttatgtctattgctggtctcggtacccggggatcgcggccgccagtgtgatggatatctgcagaattcgcccttgctggcagaacaccacaatatgactggcattgccagtgctgccacgtcggtattgatcggctatctggcggcgaataccaccacgctgcatctggggtctggcggcgtgatgttgcctaaccactcaccgttggtcattgcagaacagttcggcacgcttaatacactctatccggggcgaatcgatttggggctgggtcgtgctccgggtagtgaccaacggacaatgatggcgctacgtcgtcatatgagcggcgatattgataatttcccccgcgatgtggcggagctggtggactggtttgacgcccgcgatcccaatccgcatgtgcgcccggtaccaggctatggcgagaaaatccccgtgtggttgttaggctccagcctttacagcgcgcaactggcggcgcagcttggtctgccgtttgcgtttgcctcacacttcgcgccggatatgctgttccaggcgctgcatctttatcgcagcaacttcaaaccgtcagcacggctggaaaaaccatacgcgatggtgtgcatcaatattatcgccgccgacagcaaccgcgacgctgaatttctgtttacctcaatgcagcaagcctttgtgaagctgcgccgtggcgaaaccgggcaactgccgccgccgattcaaaatatggatcagttctggtcaccgtctgagcagtatggcgtgcagcaggcgctgagtatgtcgttggtaggtgataaagcgaaagtgcgtcatggcttgcagtcgatcctgcgcgaaaccgacgccgatgagattatggtcaacgggcagattttcgaccaccaggcgcggctgcattcgtttgagctggcgatggatgttaaggaagagttgttgggatagtgtgtcttaacgcgggaagccttatccgagctggcaacgctgtcctacatagacctgataagcgaagcgcatcaggcattgtgtaggcagcagaaatgtcggataaggcaccgctgattactctagattcgcgagggcttctctccagtgaaaaatagtgcgactgcgttgttatgcattgcactgtaccagtacacgagtacaaaagacagaaaaaaagccccgatggtaaaaatcggggctgtatatattattttacagattgtgttcgctgttcagcgatgattacgcatcaccaccgaaacgacgacgaccggtagaatcatcacgacgcggagcgcggccttcacgacgttcgccgctaaaacgacgaccatcaccacggccaccttcacggcgttcaccgctgaagttacgaccgccttcacgacgttcgccaccgaaaccacgaccaccgccacgacgctcaccgccagtatgcggctgtgcatcgcccagtaactgcatgttcatcggcttgttgagaatgcgagtgcgcgtaaagtgttgcagcacttcacccggcatacctttcggcagttcgatggtggagtgagaagcaaacagcttgatgttaccaatgtaacggctgctgatgtcgccttcgttagcaatcgcaccaacgatatgacgaacttcaacaccatcatcgcggcccacttcaatgcggtacagctgcatatcgccaacatcacgacgttcacgacgcggacgatcttcacggtcaccacgcgggccacggtcgttacgatcgcgcggaccacggtcatcacggtcacggaattcacgtttcggacgcatcggcgcatctggcggtacgatcagagtacgttcaccctgtgccattttcagcagtgccgcagccagagtttcgagatccagctcttcaccttcagcagtcggctgaattttgctcagcagtgcgcggtattgatccagatcgctgctttccagctgctgctgtactttagcggcgaatttttccagacggcgtttgcctagcagttctgcgttcggcagttctacttccggaatagtcagcttcatagtacgttcaatgttgcgcagcagacgacgctcgcggttctcaaaagggcgaattccagcacactggcggccgttactagagtcgaccggtggcgaatgggacgcgccctgtagcggcgcattaa

SEQ ID NO: 15 - DNA, 26 bp, Artificial Sequence
  misc_feature (1)..(26): Primer cmr-for
cgtcgcgatacaggcaagtcgttgag

SEQ ID NO: 16 - DNA, 26 bp, Artificial Sequence
  misc_feature (1)..(26): Primer cmr-rev
cctcgcgacagattgacgaccatcac

SEQ ID NO: 17 - DNA, 18 bp, Artificial Sequence
  misc_feature (1)..(18): Primer M13uni(-21)
tgtaaaacgacggccagt

SEQ ID NO: 18 - DNA, 18 bp, Artificial Sequence
  misc_feature (1)..(18): Primer M13rev(-29)
caggaaacagctatgacc

SEQ ID NO: 19 - DNA, 1562 bp, Escherichia coli
  misc_feature (1)..(1562): mdfA CDS with promoter and terminator
cgtcgcgatacaggcaagtcgttgagaataaagtgcaggttaaactgggtaaagcggcatcgtcttatttccctcaagcggcctgtttacggtgggtgattgtaacgggcataggttaaataaaacttaaagaaagcgtagctatactcgtaataatgtaagaatgtgcttaaccgtggtttcagctacaaaattcgctttctcgttagctgcgcttttattaaactctgcgcgattattattggcgaagaaattgcatgcaaaataaattagcttccggtgccaggcttggacgtcaggcgttacttttccctctctgtctggtgctttacgaattttcaacctatatcggcaacgatatgattcaacccggtatgttggccgtggtggaacaatatcaggcgggcattgattgggttcctacttcgatgaccgcgtatctggcgggcgggatgtttttacaatggctgctggggccgctgtcggatcgtattggtcgccgtccggtgatgctggcgggagtggtgtggtttatcgtcacctgtctggcaatattgctggcgcaaaacattgaacaattcaccctgttgcgcttcttgcagggcataagcctctgtttcattggcgctgtgggatacgccgcaattcaggaatccttcgaagaggcggtttgtatcaagatcaccgcgctgatggcgaacgtggcgctgattgctccgctacttggtccgctggtgggcgcggcgtggatccatgtgctgccctgggaggggatgtttgttttgtttgccgcattggcagcgatctcctttttcggtctgcaacgagccatgcctgaaaccgccacgcgtataggcgagaaactgtcactgaaagaactcggtcgtgactataagctggtgctgaagaacggccgctttgtggcgggggcgctggcgctgggattcgttagtctgccgttgctggcgtggatcgcccagtcgccgattatcatcattaccggcgagcagttgagcagctatgaatatggcttgctgcaagtgcctattttcggggcgttaattgcgggtaacttgctgttagcgcgtctgacctcgcgccgcaccgtacgttcgctgattattatgggcggctggccgattatgattggtctattggtcgctgctgcggcaacggttatctcatcgcacgcgtatttatggatgactgccgggttaagtatttatgctttcggtattggtctggcgaatgcgggactggtgcgattaaccctgtttgccagcgatatgagtaaaggtacggtttctgccgcgatgggaatgctgcaaatgctgatctttaccgttggtattgaaatcagcaaacatgcctggctgaacgggggcaacggactgtttaatctcttcaaccttgtcaacggaattttgtggctgtcgctgatggttatctttttaaaagataaacagatgggaaattctcacgaagggtaaaaaaatgcctgactgctttgtgcgatcaggcattctcgaattaatggtgatggtcgtcaatctgtcgcgagg

SEQ ID NO: 20 - DNA, 3120 bp, Escherichia coli
  misc_feature (1)..(3120): DaroP::mdfA; construct for integration of mdfA in delta aroP
agggcagggtcgttaaatagccgcttatgtctattgctggtctcggtacccggggatcgcggccgcggaccggatcctctagacctggcgacgaagcaacaagcccttcgcttcgagacgttgaatcgcctcacgcaaggagggacgggagacgtcaaactgttttgccagttcgcgttccggtgggagtttttcgcccgggcggagagtgccttcgaggatcaaaaactccagttgctgctcaatcacatcggagagttttggttggcggattttgctgtaggccatgagttcctgtcttaagccacttgccgaagtcaattggtcttaccaatttcatgtctgtgacgctaaagtaacaaagtattcaccttatgtccatacaggttttgattgaaatcatgaaactgtgcacattttaacaacttgacatatataacgtttcaaagttgtaactatgcacaaatgtagactttacgtaggaaaggagttttttaacgattattaaactataaaaatccgaattgaaccgattcacttaccaattttgtgatttttaattcaattaaaacgaatttaaattcattctacatattgagaggggttgaggctgagctttacaaacggtttctttttaagcaactcatcttcaaccatgcataaagcgggtgcattcgctgccgcataccattattcttgatctgacggaagtctttttgtaacaattcaaacttctttgatgtaaacaaattaatacaacaaacggaattgcaaacttacacacgcatcactgcgtagatcaaaaaaacaaccaccgcacgaggttcgcgatacaggcaagtcgttgagaataaagtgcaggttaaactgggtaaagcggcatcgtcttatttccctcaagcggcctgtttacggtgggtgattgtaacgggcataggttaaataaaacttaaagaaagcgtagctatactcgtaataatgtaagaatgtgcttaaccgtggtttcagctacaaaattcgctttctcgttagctgcgcttttattaaactctgcgcgattattattggcgaagaaattgcatgcaaaataaattagcttccggtgccaggcttggacgtcaggcgttacttttccctctctgtctggtgctttacgaattttcaacctatatcggcaacgatatgattcaacccggtatgttggccgtggtggaacaatatcaggcgggcattgattgggttcctacttcgatgaccgcgtatctggcgggcgggatgtttttacaatggctgctggggccgctgtcggatcgtattggtcgccgtccggtgatgctggcgggagtggtgtggtttatcgtcacctgtctggcaatattgctggcgcaaaacattgaacaattcaccctgttgcgcttcttgcagggcataagcctctgtttcattggcgctgtgggatacgccgcaattcaggaatccttcgaagaggcggtttgtatcaagatcaccgcgctgatggcgaacgtggcgctgattgctccgctacttggtccgctggtgggcgcggcgtggatccatgtgctgccctgggaggggatgtttgttttgtttgccgcattggcagcgatctcctttttcggtctgcaacgagccatgcctgaaaccgccacgcgtataggcgagaaactgtcactgaaagaactcggtcgtgactataagctggtgctgaagaacggccgctttgtggcgggggcgctggcgctgggattcgttagtctgccgttgctggcgtggatcgcccagtcgccgattatcatcattaccggcgagcagttgagcagctatgaatatggcttgctgcaagtgcctattttcggggcgttaattgcgggtaacttgctgttagcgcgtctgacctcgcgccgcaccgtacgttcgctgattattatgggcggctggccgattatgattggtctattggtcgctgctgcggcaacggttatctcatcgcacgcgtatttatggatgactgccgggttaagtatttatgctttcggtattggtctggcgaatgcgggactggtgcgattaaccctgtttgccagcgatatgagtaaaggtacggtttctgccgcgatgggaatgctgcaaatgctgatctttaccgttggtattgaaatcagcaaacatgcctggctgaacgggggcaacggactgtttaatctcttcaaccttgtcaacggaattttgtggctgtcgctgatggttatctttttaaaagataaacagatgggaaattctcacgaagggtaaaaaaatgcctgactgctttgtgcgatcaggcattctcgaattaatggtgatggtcgtcaatctgtcgcgattacaccaacgccccgtaaatcgtcagtagtgcaatcaccaccacgaccacgaacgaggttttcttcgccattgaaaccgctgccttcggcgtctccaccttatcgacatgcggttcacgcgccagagagaactgcgccagacgcgttaacacctgatactgcgaagtatggaaatcacccagcgaagcaaaccaggccggtaacgctttctcaccatgaccgatcaaggcatataccacacccgcaagacgaaccggcacccaatccagtacatgaagcacggcatcaatgccggactgtaaacgatgatgcggcgtctgatatcgcgccagccagtattgccatgcacgcaaaaacgcataccccatcagcgtaacgggtccccaggttccccccacaatcagccagaacagcggtgcaagataaaaacgaaagttaatccacagcaatgcattttgcagctcacgcaaatactcacgttcgtcgcagcctgccgggacgccgtgaatcatggtgagttcgccagccatcgtggcacgggcatggctatcattacgtgaagcagctgtcagataagcatgataatgaagacgaactttacctgcgccaatacacagcaaaccaatcagcagccacaccagtagcgtgggaacgttgaacaatactccctgcaatgcgcgtaacagtctagagtcgaccggtggcgaatgggacgcgccctgtagcggcgcattaa

SEQ ID NO: 21 - DNA, 3735 bp, Escherichia coli
  misc_feature (1)..(3735): Dmtr::mdfA; construct for integration of mdfA in delta mtr
agggcagggtcgttaaatagccgcttatgtctattgctggtctcggtacccggggatcgcggccgccagtgtgatggatatctgcagaattcgcccttgctggcagaacaccacaatatgactggcattgccagtgctgccacgtcggtattgatcggctatctggcggcgaataccaccacgctgcatctggggtctggcggcgtgatgttgcctaaccactcaccgttggtcattgcagaacagttcggcacgcttaatacactctatccggggcgaatcgatttggggctgggtcgtgctccgggtagtgaccaacggacaatgatggcgctacgtcgtcatatgagcggcgatattgataatttcccccgcgatgtggcggagctggtggactggtttgacgcccgcgatcccaatccgcatgtgcgcccggtaccaggctatggcgagaaaatccccgtgtggttgttaggctccagcctttacagcgcgcaactggcggcgcagcttggtctgccgtttgcgtttgcctcacacttcgcgccggatatgctgttccaggcgctgcatctttatcgcagcaacttcaaaccgtcagcacggctggaaaaaccatacgcgatggtgtgcatcaatattatcgccgccgacagcaaccgcgacgctgaatttctgtttacctcaatgcagcaagcctttgtgaagctgcgccgtggcgaaaccgggcaactgccgccgccgattcaaaatatggatcagttctggtcaccgtctgagcagtatggcgtgcagcaggcgctgagtatgtcgttggtaggtgataaagcgaaagtgcgtcatggcttgcagtcgatcctgcgcgaaaccgacgccgatgagattatggtcaacgggcagattttcgaccaccaggcgcggctgcattcgtttgagctggcgatggatgttaaggaagagttgttgggatagtgtgtcttaacgcgggaagccttatccgagctggcaacgctgtcctacatagacctgataagcgaagcgcatcaggcattgtgtaggcagcagaaatgtcggataaggcaccgctgattactctagattcgcgacagattgacgaccatcaccattaattcgagaatgcctgatcgcacaaagcagtcaggcatttttttacccttcgtgagaatttcccatctgtttatcttttaaaaagataaccatcagcgacagccacaaaattccgttgacaaggttgaagagattaaacagtccgttgcccccgttcagccaggcatgtttgctgatttcaataccaacggtaaagatcagcatttgcagcattcccatcgcggcagaaaccgtacctttactcatatcgctggcaaacagggttaatcgcaccagtcccgcattcgccagaccaataccgaaagcataaatacttaacccggcagtcatccataaatacgcgtgcgatgagataaccgttgccgcagcagcgaccaatagaccaatcataatcggccagccgcccataataatcagcgaacgtacggtgcggcgcgaggtcagacgcgctaacagcaagttacccgcaattaacgccccgaaaataggcacttgcagcaagccatattcatagctgctcaactgctcgccggtaatgatgataatcggcgactgggcgatccacgccagcaacggcagactaacgaatcccagcgccagcgcccccgccacaaagcggccgttcttcagcaccagcttatagtcacgaccgagttctttcagtgacagtttctcgcctatacgcgtggcggtttcaggcatggctcgttgcagaccgaaaaaggagatcgctgccaatgcggcaaacaaaacaaacatcccctcccagggcagcacatggatccacgccgcgcccaccagcggaccaagtagcggagcaatcagcgccacgttcgccatcagcgcggtgatcttgatacaaaccgcctcttcgaaggattcctgaattgcggcgtatcccacagcgccaatgaaacagaggcttatgccctgcaagaagcgcaacagggtgaattgttcaatgttttgcgccagcaatattgccagacaggtgacgataaaccacaccactcccgccagcatcaccggacggcgaccaatacgatccgacagcggccccagcagccattgtaaaaacatcccgcccgccagatacgcggtcatcgaagtaggaacccaatcaatgcccgcctgatattgttccaccacggccaacataccgggttgaatcatatcgttgccgatataggttgaaaattcgtaaagcaccagacagagagggaaaagtaacgcctgacgtccaagcctggcaccggaagctaatttattttgcatgcaatttcttcgccaataataatcgcgcagagtttaataaaagcgcagctaacgagaaagcgaattttgtagctgaaaccacggttaagcacattcttacattattacgagtatagctacgctttctttaagttttatttaacctatgcccgttacaatcacccaccgtaaacaggccgcttgagggaaataagacgatgccgctttacccagtttaacctgcactttattctcaacgacttgcctgtatcgcgagggcttctctccagtgaaaaatagtgcgactgcgttgttatgcattgcactgtaccagtacacgagtacaaaagacagaaaaaaagccccgatggtaaaaatcggggctgtatatattattttacagattgtgttcgctgttcagcgatgattacgcatcaccaccgaaacgacgacgaccggtagaatcatcacgacgcggagcgcggccttcacgacgttcgccgctaaaacgacgaccatcaccacggccaccttcacggcgttcaccgctgaagttacgaccgccttcacgacgttcgccaccgaaaccacgaccaccgccacgacgctcaccgccagtatgcggctgtgcatcgcccagtaactgcatgttcatcggcttgttgagaatgcgagtgcgcgtaaagtgttgcagcacttcacccggcatacctttcggcagttcgatggtggagtgagaagcaaacagcttgatgttaccaatgtaacggctgctgatgtcgccttcgttagcaatcgcaccaacgatatgacgaacttcaacaccatcatcgcggcccacttcaatgcggtacagctgcatatcgccaacatcacgacgttcacgacgcggacgatcttcacggtcaccacgcgggccacggtcgttacgatcgcgcggaccacggtcatcacggtcacggaattcacgtttcggacgcatcggcgcatctggcggtacgatcagagtacgttcaccctgtgccattttcagcagtgccgcagccagagtttcgagatccagctcttcaccttcagcagtcggctgaattttgctcagcagtgcgcggtattgatccagatcgctgctttccagctgctgctgtactttagcggcgaatttttccagacggcgtttgcctagcagttctgcgttcggcagttctacttccggaatagtcagcttcatagtacgttcaatgttgcgcagcagacgacgctcgcggttctcaaaagggcgaattccagcacactggcggccgttactagagtcgaccggtggcgaatgggacgcgccctgtagcggcgcattaa

SEQ ID NO: 22 - DNA, 1436 bp, Escherichia coli
  misc_feature (1)..(1436): delta aroP on chromosome
cctggcgacgaagcaacaagcccttcgcttcgagacgttgaatcgcctcacgcaaggagggacgggagacgtcaaactgttttgccagttcgcgttccggtgggagtttttcgcccgggcggagagtgccttcgaggatcaaaaactccagttgctgctcaatcacatcggagagttttggttggcggattttgctgtaggccatgagttcctgtcttaagccacttgccgaagtcaattggtcttaccaatttcatgtctgtgacgctaaagtaacaaagtattcaccttatgtccatacaggttttgattgaaatcatgaaactgtgcacattttaacaacttgacatatataacgtttcaaagttgtaactatgcacaaatgtagactttacgtaggaaaggagttttttaacgattattaaactataaaaatccgaattgaaccgattcacttaccaattttgtgatttttaattcaattaaaacgaatttaaattcattctacatattgagaggggttgaggctgagctttacaaacggtttctttttaagcaactcatcttcaaccatgcataaagcgggtgcattcgctgccgcataccattattcttgatctgacggaagtctttttgtaacaattcaaacttctttgatgtaaacaaattaatacaacaaacggaattgcaaacttacacacgcatcactgcgtagatcaaaaaaacaaccaccgcacgaggttcgcgattacaccaacgccccgtaaatcgtcagtagtgcaatcaccaccacgaccacgaacgaggttttcttcgccattgaaaccgctgccttcggcgtctccaccttatcgacatgcggttcacgcgccagagagaactgcgccagacgcgttaacacctgatactgcgaagtatggaaatcacccagcgaagcaaaccaggccggtaacgctttctcaccatgaccgatcaaggcatataccacacccgcaagacgaaccggcacccaatccagtacatgaagcacggcatcaatgccggactgtaaacgatgatgcggcgtctgatatcgcgccagccagtattgccatgcacgcaaaaacgcataccccatcagcgtaacgggtccccaggttccccccacaatcagccagaacagcggtgcaagataaaaacgaaagttaatccacagcaatgcattttgcagctcacgcaaatactcacgttcgtcgcagcctgccgggacgccgtgaatcatggtgagttcgccagccatcgtggcacgggcatggctatcattacgtgaagcagctgtcagataagcatgataatgaagacgaactttacctgcgccaatacacagcaaaccaatcagcagccacaccagtagcgtgggaacgttgaacaatactccctgcaatgcgcgtaacagt

SEQ ID NO: 23 - DNA, 2988 bp, Escherichia coli
  misc_feature (1)..(2988): mdfA in delta aroP on chromosome
cctggcgacgaagcaacaagcccttcgcttcgagacgttgaatcgcctcacgcaaggagggacgggagacgtcaaactgttttgccagttcgcgttccggtgggagtttttcgcccgggcggagagtgccttcgaggatcaaaaactccagttgctgctcaatcacatcggagagttttggttggcggattttgctgtaggccatgagttcctgtcttaagccacttgccgaagtcaattggtcttaccaatttcatgtctgtgacgctaaagtaacaaagtattcaccttatgtccatacaggttttgattgaaatcatgaaactgtgcacattttaacaacttgacatatataacgtttcaaagttgtaactatgcacaaatgtagactttacgtaggaaaggagttttttaacgattattaaactataaaaatccgaattgaaccgattcacttaccaattttgtgatttttaattcaattaaaacgaatttaaattcattctacatattgagaggggttgaggctgagctttacaaacggtttctttttaagcaactcatcttcaaccatgcataaagcgggtgcattcgctgccgcataccattattcttgatctgacggaagtctttttgtaacaattcaaacttctttgatgtaaacaaattaatacaacaaacggaattgcaaacttacacacgcatcactgcgtagatcaaaaaaacaaccaccgcacgaggttcgcgatacaggcaagtcgttgagaataaagtgcaggttaaactgggtaaagcggcatcgtcttatttccctcaagcggcctgtttacggtgggtgattgtaacgggcataggttaaataaaacttaaagaaagcgtagctatactcgtaataatgtaagaatgtgcttaaccgtggtttcagctacaaaattcgctttctcgttagctgcgcttttattaaactctgcgcgattattattggcgaagaaattgcatgcaaaataaattagcttccggtgccaggcttggacgtcaggcgttacttttccctctctgtctggtgctttacgaattttcaacctatatcggcaacgatatgattcaacccggtatgttggccgtggtggaacaatatcaggcgggcattgattgggttcctacttcgatgaccgcgtatctggcgggcgggatgtttttacaatggctgctggggccgctgtcggatcgtattggtcgccgtccggtgatgctggcgggagtggtgtggtttatcgtcacctgtctggcaatattgctggcgcaaaacattgaacaattcaccctgttgcgcttcttgcagggcataagcctctgtttcattggcgctgtgggatacgccgcaattcaggaatccttcgaagaggcggtttgtatcaagatcaccgcgctgatggcgaacgtggcgctgattgctccgctacttggtccgctggtgggcgcggcgtggatccatgtgctgccctgggaggggatgtttgttttgtttgccgcattggcagcgatctcctttttcggtctgcaacgagccatgcctgaaaccgccacgcgtataggcgagaaactgtcactgaaagaactcggtcgtgactataagctggtgctgaagaacggccgctttgtggcgggggcgctggcgctgggattcgttagtctgccgttgctggcgtggatcgcccagtcgccgattatcatcattaccggcgagcagttgagcagctatgaatatggcttgctgcaagtgcctattttcggggcgttaattgcgggtaacttgctgttagcgcgtctgacctcgcgccgcaccgtacgttcgctgattattatgggcggctggccgattatgattggtctattggtcgctgctgcggcaacggttatctcatcgcacgcgtatttatggatgactgccgggttaagtatttatgctttcggtattggtctggcgaatgcgggactggtgcgattaaccctgtttgccagcgatatgagtaaaggtacggtttctgccgcgatgggaatgctgcaaatgctgatctttaccgttggtattgaaatcagcaaacatgcctggctgaacgggggcaacggactgtttaatctcttcaaccttgtcaacggaattttgtggctgtcgctgatggttatctttttaaaagataaacagatgggaaattctcacgaagggtaaaaaaatgcctgactgctttgtgcgatcaggcattctcgaattaatggtgatggtcgtcaatctgtcgcgattacaccaacgccccgtaaatcgtcagtagtgcaatcaccaccacgaccacgaacgaggttttcttcgccattgaaaccgctgccttcggcgtctccaccttatcgacatgcggttcacgcgccagagagaactgcgccagacgcgttaacacctgatactgcgaagtatggaaatcacccagcgaagcaaaccaggccggtaacgctttctcaccatgaccgatcaaggcatataccacacccgcaagacgaaccggcacccaatccagtacatgaagcacggcatcaatgccggactgtaaacgatgatgcggcgtctgatatcgcgccagccagtattgccatgcacgcaaaaacgcataccccatcagcgtaacgggtccccaggttccccccacaatcagccagaacagcggtgcaagataaaaacgaaagttaatccacagcaatgcattttgcagctcacgcaaatactcacgttcgtcgcagcctgccgggacgccgtgaatcatggtgagttcgccagccatcgtggcacgggcatggctatcattacgtgaagcagctgtcagataagcatgataatgaagacgaactttacctgcgccaatacacagcaaaccaatcagcagccacaccagtagcgtgggaacgttgaacaatactccctgcaatgcgcgtaacagt

SEQ ID NO: 24 - DNA, 2004 bp, Escherichia coli
  misc_feature (1)..(2004): delta mtr on chromosome
gctggcagaacaccacaatatgactggcattgccagtgctgccacgtcggtattgatcggctatctggcggcgaataccaccacgctgcatctggggtctggcggcgtgatgttgcctaaccactcaccgttggtcattgcagaacagttcggcacgcttaatacactctatccggggcgaatcgatttggggctgggtcgtgctccgggtagtgaccaacggacaatgatggcgctacgtcgtcatatgagcggcgatattgataatttcccccgcgatgtggcggagctggtggactggtttgacgcccgcgatcccaatccgcatgtgcgcccggtaccaggctatggcgagaaaatccccgtgtggttgttaggctccagcctttacagcgcgcaactggcggcgcagcttggtctgccgtttgcgtttgcctcacacttcgcgccggatatgctgttccaggcgctgcatctttatcgcagcaacttcaaaccgtcagcacggctggaaaaaccatacgcgatggtgtgcatcaatattatcgccgccgacagcaaccgcgacgctgaatttctgtttacctcaatgcagcaagcctttgtgaagctgcgccgtggcgaaaccgggcaactgccgccgccgattcaaaatatggatcagttctggtcaccgtctgagcagtatggcgtgcagcaggcgctgagtatgtcgttggtaggtgataaagcgaaagtgcgtcatggcttgcagtcgatcctgcgcgaaaccgacgccgatgagattatggtcaacgggcagattttcgaccaccaggcgcggctgcattcgtttgagctggcgatggatgttaaggaagagttgttgggatagtgtgtcttaacgcgggaagccttatccgagctggcaacgctgtcctacatagacctgataagcgaagcgcatcaggcattgtgtaggcagcagaaatgtcggataaggcaccgctgattactctagattcgcgagggcttctctccagtgaaaaatagtgcgactgcgttgttatgcattgcactgtaccagtacacgagtacaaaagacagaaaaaaagccccgatggtaaaaatcggggctgtatatattattttacagattgtgttcgctgttcagcgatgattacgcatcaccaccgaaacgacgacgaccggtagaatcatcacgacgcggagcgcggccttcacgacgttcgccgctaaaacgacgaccatcaccacggccaccttcacggcgttcaccgctgaagttacgaccgccttcacgacgttcgccaccgaaaccacgaccaccgccacgacgctcaccgccagtatgcggctgtgcatcgcccagtaactgcatgttcatcggcttgttgagaatgcgagtgcgcgtaaagtgttgcagcacttcacccggcatacctttcggcagttcgatggtggagtgagaagcaaacagcttgatgttaccaatgtaacggctgctgatgtcgccttcgttagcaatcgcaccaacgatatgacgaacttcaacaccatcatcgcggcccacttcaatgcggtacagctgcatatcgccaacatcacgacgttcacgacgcggacgatcttcacggtcaccacgcgggccacggtcgttacgatcgcgcggaccacggtcatcacggtcacggaattcacgtttcggacgcatcggcgcatctggcggtacgatcagagtacgttcaccctgtgccattttcagcagtgccgcagccagagtttcgagatccagctcttcaccttcagcagtcggctgaattttgctcagcagtgcgcggtattgatccagatcgctgctttccagctgctgctgtactttagcggcgaatttttccagacggcgtttgcctagcagttctgcgttcggcagttctacttccggaatagtcagcttcatagtacgttcaatgttgcgcagcagacgacgctcgcggttctcaa

SEQ ID NO: 25 - DNA, 3553 bp, Escherichia coli
  misc_feature (1)..(3553): mdfA in delta mtr on chromosome
ctggcagaacaccacaatatgactggcattgccagtgctgccacgtcggtattgatcggctatctggcggcgaataccaccacgctgcatctggggtctggcggcgtgatgttgcctaaccactcaccgttggtcattgcagaacagttcggcacgcttaatacactctatccggggcgaatcgatttggggctgggtcgtgctccgggtagtgaccaacggacaatgatggcgctacgtcgtcatatgagcggcgatattgataatttcccccgcgatgtggcggagctggtggactggtttgacgcccgcgatcccaatccgcatgtgcgcccggtaccaggctatggcgagaaaatccccgtgtggttgttaggctccagcctttacagcgcgcaactggcggcgcagcttggtctgccgtttgcgtttgcctcacacttcgcgccggatatgctgttccaggcgctgcatctttatcgcagcaacttcaaaccgtcagcacggctggaaaaaccatacgcgatggtgtgcatcaatattatcgccgccgacagcaaccgcgacgctgaatttctgtttacctcaatgcagcaagcctttgtgaagctgcgccgtggcgaaaccgggcaactgccgccgccgattcaaaatatggatcagttctggtcaccgtctgagcagtatggcgtgcagcaggcgctgagtatgtcgttggtaggtgataaagcgaaagtgcgtcatggcttgcagtcgatcctgcgcgaaaccgacgccgatgagattatggtcaacgggcagattttcgaccaccaggcgcggctgcattcgtttgagctggcgatggatgttaaggaagagttgttgggatagtgtgtcttaacgcgggaagccttatccgagctggcaacgctgtcctacatagacctgataagcgaagcgcatcaggcattgtgtaggcagcagaaatgtcggataaggcaccgctgattactctagattcgcgacagattgacgaccatcaccattaattcgagaatgcctgatcgcacaaagcagtcaggcatttttttacccttcgtgagaatttcccatctgtttatcttttaaaaagataaccatcagcgacagccacaaaattccgttgacaaggttgaagagattaaacagtccgttgcccccgttcagccaggcatgtttgctgatttcaataccaacggtaaagatcagcatttgcagcattcccatcgcggcagaaaccgtacctttactcatatcgctggcaaacagggttaatcgcaccagtcccgcattcgccagaccaataccgaaagcataaatacttaacccggcagtcatccataaatacgcgtgcgatgagataaccgttgccgcagcagcgaccaatagaccaatcataatcggccagccgcccataataatcagcgaacgtacggtgcggcgcgaggtcagacgcgctaacagcaagttacccgcaattaacgccccgaaaataggcacttgcagcaagccatattcatagctgctcaactgctcgccggtaatgatgataatcggcgactgggcgatccacgccagcaacggcagactaacgaatcccagcgccagcgcccccgccacaaagcggccgttcttcagcaccagcttatagtcacgaccgagttctttcagtgacagtttctcgcctatacgcgtggcggtttcaggcatggctcgttgcagaccgaaaaaggagatcgctgccaatgcggcaaacaaaacaaacatcccctcccagggcagcacatggatccacgccgcgcccaccagcggaccaagtagcggagcaatcagcgccacgttcgccatcagcgcggtgatcttgatacaaaccgcctcttcgaaggattcctgaattgcggcgtatcccacagcgccaatgaaacagaggcttatgccctgcaagaagcgcaacagggtgaattgttcaatgttttgcgccagcaatattgccagacaggtgacgataaaccacaccactcccgccagcatcaccggacggcgaccaatacgatccgacagcggccccagcagccattgtaaaaacatcccgcccgccagatacgcggtcatcgaagtaggaacccaatcaatgcccgcctgatattgttccaccacggccaacataccgggttgaatcatatcgttgccgatataggttgaaaattcgtaaagcaccagacagagagggaaaagtaacgcctgacgtccaagcctggcaccggaagctaatttattttgcatgcaatttcttcgccaataataatcgcgcagagtttaataaaagcgcagctaacgagaaagcgaattttgtagctgaaaccacggttaagcacattcttacattattacgagtatagctacgctttctttaagttttatttaacctatgcccgttacaatcacccaccgtaaacaggccgcttgagggaaataagacgatgccgctttacccagtttaacctgcactttattctcaacgacttgcctgtatcgcgagggcttctctccagtgaaaaatagtgcgactgcgttgttatgcattgcactgtaccagtacacgagtacaaaagacagaaaaaaagccccgatggtaaaaatcggggctgtatatattattttacagattgtgttcgctgttcagcgatgattacgcatcaccaccgaaacgacgacgaccggtagaatcatcacgacgcggagcgcggccttcacgacgttcgccgctaaaacgacgaccatcaccacggccaccttcacggcgttcaccgctgaagttacgaccgccttcacgacgttcgccaccgaaaccacgaccaccgccacgacgctcaccgccagtatgcggctgtgcatcgcccagtaactgcatgttcatcggcttgttgagaatgcgagtgcgcgtaaagtgttgcagcacttcacccggcatacctttcggcagttcgatggtggagtgagaagcaaacagcttgatgttaccaatgtaacggctgctgatgtcgccttcgttagcaatcgcaccaacgatatgacgaacttcaacaccatcatcgcggcccacttcaatgcggtacagctgcatatcgccaacatcacgacgttcacgacgcggacgatcttcacggtcaccacgcgggccacggtcgttacgatcgcgcggaccacggtcatcacggtcacggaattcacgtttcggacgcatcggcgcatctggcggtacgatcagagtacgttcaccctgtgccattttcagcagtgccgcagccagagtttcgagatccagctcttcaccttcagcagtcggctgaattttgctcagcagtgcgcggtattgatccagatcgctgctttccagctgctgctgtactttagcggcgaatttttccagacggcgtttgcctagcagttctgcgttcggcagttctacttccggaatagtcagcttcatagtacgttcaatgttgcgcagcagacgacgctcgcggttctc